(19) United States
(12) Patent Application Publication
(10) Pub. No.: US 2025/0374896 A1
(43) Pub. Date: Dec. 11, 2025

(54) ANIMAL MODELS AND THERAPEUTIC MOLECULES

(71) Applicant: PETMEDIX LTD., Cambridge (GB)

(72) Inventors: Albert Vilella BERTRAN, Cambridge (GB); Andreas MARQUARDT, Cambridge (GB)

(21) Appl. No.: 18/876,826

(22) PCT Filed: Jun. 23, 2023
(86) PCT No.: PCT/EP2023/067184

§ 371 Date: Dec. 19, 2024

(30) Foreign Application Priority Data
Jun. 23, 2022 (GB) 2209247.2

(51) Int. Cl. A01K 67/0278; C07K 16/46

(52) U.S. Cl. CPC A01K 67/0278; C07K 16/462; A01K 2217/072; A01K 2227/105; A01K 2267/01

(57) ABSTRACT

The present invention relates inter alia to rodents and cells that are engineered to contain the exogenous DNA of cats, their use in medicine and the study of disease, methods for production of rodents and cells, and antibodies and antibody chains produced by such rodents and derivatives thereof.

### BACKGROUND

The present invention relates inter alia to rodents and cells that are engineered to contain the exogenous DNA of cats, their use in medicine and the study of disease, methods for production of rodents and cells, and antibodies and antibody chains produced by such rodents and derivatives thereof.

Insertion of human DNA into rodents has been disclosed in e.g. Murphy et al, Vol 111 no 14, 5153-5158, doi: 10.1073/pnas. 1324022111; MacDonald et al vol. 111 no. 14, 5147-5152, doi: 10.1073/pnas. 1323896111; and Lee et al, Nature Biotechnology Volume: 32, Pages: 356-363 2014 DOI:, doi: 10.1038/nbt.2825. This approach is designed to make antibody products for human therapeutic use. Insertion of dog and cat DNA into rodents has been described in WO2018189520.

The present invention relates to rodents and cells as well as antibody repertoires, antibodies and parts of antibodies, such as those produced from rodents comprising cat immunoglobulin DNA, including fully cat antibodies, and use of such antibodies and parts thereof in cats for prevention and treatment of disease, as well as methods for the manufacture of such rodents, cells, antibodies, antibody chains and repertoires.

### STATEMENTS OF INVENTION

A rodent or rodent cell having a genome comprising:
i) no more than 9 cat IGHV gene segments, one or more cat IGHD region gene segment(s) and one or more cat IGHJ region gene segment(s),
wherein the rodent or rodent cell is capable of expressing the cat IGH gene segments to form an antibody heavy chain,
   wherein at least one of the cat IGH V gene segments is selected from the list comprising V3-14, V3-11, V3-12, V3-84, V3-20, V3-27, V4-3, V3-N3 and V3-5.

A rodent or rodent cell having a genome comprising:
i) no more than 11 cat lambda V gene segments and one or more cat IGL J region gene segment(s), wherein the rodent or rodent cell is capable of expressing the cat lambda gene segments to form an antibody light chain,
   wherein at least one of the cat lambda gene segments is selected from the list comprising V1-124, V1-36, V1-104, V1-33, V1-56, V1-42, V1-41, V3-7, V3-5, V3-13 and V1-126.

A rodent or rodent cell having a genome comprising;
i) no more than 6 cat kappa V gene segments and one or more cat IGKJ region gene segment(s);
wherein the rodent or rodent cell is capable of expressing the cat kappa gene segments to form an antibody light chain,
   wherein at least one of the cat kappa gene segments is selected from V4-1, V2-12, V2-5, V2-13, V2-9 and V2-4.

In an embodiment, the gene segments have a nucleic acid sequence as set out in Table 1, where “V3-14”, “V-124” and so forth are designated corresponding SEQ ID NOs. By “gene segments” is also meant “gene”.

A method for producing a rodent or rodent cell disclosed herein, the method comprising inserting into a rodent cell genome
(i) no more than 9 cat IGHV gene segments, one or more cat IGH D region gene segments and one or more cat IGH J region gene segments, wherein at least one of the cat IGH V gene segments is selected from the list comprising V3-14, V3-11, V3-12, V3-84, V3-20, V3-27, V4-3, V3-N3 and V3-5; and/or
(ii) no more than 11 cat lambda V gene segments and one or more cat lambda J region gene segments, wherein at least one of the cat lambda V gene segments is selected from the list comprising V1-124, V1-36, V1-104, V1-33, V1-56, V1-42, V1-41, V3-7, V3-5, V3-13 and V1-126; and/or
(iii) no more than 6 cat kappa V gene segments and one or more cat J region gene segments, wherein at least one of the cat kappa V gene segments is selected from V4-1, V2-12, V2-5, V2-13, V2-9 and V2-4;
and wherein the rodent or rodent cell is capable of expressing the cat variable region gene segment or segments in combination with a constant region to form an antibody chain.

References herein to cat variable region gene segments are references to cat V region gene segments, cat D region gene segments and/or cat J region gene segments as appropriate. In an aspect, the cat variable region gene segments are at least 1 cat V region gene segment, at least one cat D region gene segment and at least one cat J region gene segment.

A method for producing an antibody or antibody chain specific to a desired antigen, the method comprising immunizing a rodent disclosed herein with the desired antigen and recovering the antibody chain or antibody, or recovering a cell producing the antibody chain or antibody.

A method for producing an antibody chain or antibody specific to a desired antigen the method comprising immunizing a rodent disclosed herein and then replacing any rodent constant region of the antibody chain or antibody with a cat constant region, suitably by engineering of the nucleic acid encoding the antibody.

References herein to replacing any rodent constant region means that in situations where the constant region is a rodent constant region, it is replaced with, for example, a cat constant region, and that in situations where the constant region is already, for example, a cat constant region, no such replacement is necessary. This may be the case, for example, at the lambda locus.

A method for producing an antibody, antibody chain or a part thereof, the antibody chain having a cat variable region, the method comprising expressing in a cell a nucleic acid such as DNA encoding the antibody, antibody chain, or a part thereof,
wherein the sequence of the nucleic acid encoding the variable region of the antibody chain is obtained from immunising a rodent disclosed herein with an antigen,
optionally including the subsequent steps of:
i. purifying and/or isolating the antibody or antibody chain, and
ii. optionally then formulating the antibody or antibody chain into a pharmaceutically acceptable formulation suitable for administration into a cat.

A method of making a pharmaceutical composition, the method comprising producing an antibody, preferably a fully cat antibody, according to a method disclosed herein and further comprising combining the antibody with a pharmaceutically acceptable carrier or other excipient to produce the composition.

A nucleic acid encoding a chimaeric antibody chain, the nucleic acid comprising any one of the cat IGHV gene segments selected from the list comprising V3-14, V3-11, V3-12, V3-84, V3-20, V3-27, V4-3, V3-N3 and V3-5 together with a rodent constant region, optionally wherein the cat V gene segment is a sequence that has undergone somatic hypermutation in a rodent.

A nucleic acid encoding a chimaeric antibody chain, the nucleic acid comprising any 2, 3, 4, 5, 6, 7, 8, or all 9 of the cat IGHV gene segments selected from the list comprising V3-14, V3-11, V3-12, V3-84, V3-20, V3-27, V4-3, V3-N3 and V3-5 together with a rodent constant region, optionally wherein the cat V gene segment is a sequence that has undergone somatic hypermutation in a rodent.

A nucleic acid encoding a chimaeric antibody chain, the nucleic acid comprising any one of the cat lambda V gene segments selected from the list comprising lambda V1-124, V1-36, V1-104, V1-33, V1-56, V1-42, V1-41, V3-7, V3-5, V3-13 and V1-126 together with a rodent constant region, optionally wherein the cat V gene segment sequence is a sequence that has undergone somatic hypermutation in a rodent.

A nucleic acid encoding a chimaeric antibody chain, the nucleic acid comprising any 2, 3, 4, 5, 6, 7, 8, 9, 10, or all 11 of the cat lambda V gene segments selected from the list comprising lambda V1-124, V1-36, V1-104, V1-33, V1-56, V1-42, V1-41, V3-7, V3-5, V3-13 and V1-126 together with a rodent constant region, optionally wherein the cat V gene segment is a sequence that has undergone somatic hypermutation in a rodent.

A nucleic acid encoding a chimaeric antibody chain, the nucleic acid comprising any one of the cat kappa V gene segments selected from the list comprising V4-1, V2-12, V2-5, V2-13, V2-9 and V2-4 together with a rodent constant region, optionally wherein the cat V gene segment sequence is a sequence that has undergone somatic hypermutation in a rodent.

A nucleic acid encoding a chimaeric antibody chain, the nucleic acid comprising any 2, 3, 4, 5 or all 6 of the cat kappa V gene segments selected from the list comprising V4-1, V2-12, V2-5, V2-13, V2-9 and V2-4 together with a rodent constant region, optionally wherein the cat V gene segment is a sequence that has undergone somatic hypermutation in a rodent.

An antibody chain having a variable region obtained by expression of any one of cat IGH V3-14, V3-11, V3-12, V3-84, V3-20, V3-27, V4-3, V3-N3 and V3-5 in a rodent, in conjunction with a rodent constant region.

An antibody chain having a variable region obtained by expression of any 2, 3, 4, 5, 6, 7, 8 or all 9 of cat IGH V3-14, V3-11, V3-12, V3-84, V3-20, V3-27, V4-3, V3-N3 and V3-5 in a rodent, in conjunction with a rodent constant region.

An antibody chain having a variable region obtained by expression of any one of cat lambda V1-124, V1-36, V1-104, V1-33, V1-56, V1-42, V1-41, V3-7, V3-5, V3-13 and V1-126 in a rodent, in conjunction with a rodent constant region.

An antibody chain having a variable region obtained by expression of any 2, 3, 4, 5, 6, 7, 8, 9, 10, or all 11 of cat lambda V1-124, V1-36, V1-104, V1-33, V1-56, V1-42, V1-41, V3-7, V3-5, V3-13 and V1-126 in a rodent, in conjunction with a rodent constant region.

An antibody chain having a variable region obtained by expression of any one of cat kappa V4-1, V2-12, V2-5, V2-13, V2-9 and V2-4 in a rodent, in conjunction with a rodent constant region. An antibody chain having a variable region obtained by expression of any 2, 3, 4, 5, or all 6 of cat kappa V4-1, V2-12, V2-5, V2-13, V2-9 and V2-4 in a rodent, in conjunction with a rodent constant region.

A pharmaceutical composition comprising an antibody chain having a variable region comprising any one of, or any 2 of, 3 of, 4 of, 5 of, 6 of, 7 of, 8 of, or all 9 of cat IGH V3-14, V3-11, V3-12, V3-84, V3-20, V3-27, V4-3, V3-N3 and V3-5, in conjunction with a pharmaceutically acceptable excipient or carrier.

A pharmaceutical composition comprising an antibody chain having a variable region comprising any one of, or any 2 of, 3 of, 4 of, 5 of, 6 of, 7 of, 8 of, 9 of, 10 of, or all 11 of cat lambda V1-124, V1-36, V1-104, V1-33, V1-56, V1-42, V1-41, V3-7, V3-5, V3-13 and V1-126, in conjunction with a pharmaceutically acceptable excipient or carrier.

A pharmaceutical composition comprising an antibody chain having a variable region comprising any one of, or any 2 of, 3 of, 4 of, 5 of, or all 6 of cat kappa V4-1, V2-12, V2-5, V2-13, V2-9 and V2-4, in conjunction with a pharmaceutically acceptable excipient or carrier.

A pharmaceutical composition comprising an antibody, the antibody having
(i) a heavy chain having a variable region obtained by expression of any one of cat IGH V3-14, V3-11, V3-12, V3-84, V3-20, V3-27, V4-3, V3-N3 and V3-5, and either
(ii) a lambda light chain obtained by expression of any one of cat lambda V1-124, V1-36, V1-104, V1-33, V1-56, V1-42, V1-41, V3-7, V3-5, V3-13 and V1-126; or
(iii) a kappa light chain obtained by expression of any one of cat kappa V4-1, V2-12, V2-5, V2-13, V2-9 and V2-4;
   the antibody being formulated in conjunction with a pharmaceutically acceptable excipient or carrier.

An antibody heavy chain repertoire, the repertoire comprising antibody heavy chains having cat IGH V gene segments from no more than 9 different cat IGH V gene segments, wherein at least one of the cat IGH V gene segments is selected from the list comprising V3-14, V3-11, V3-12, V3-84, V3-20, V3-27, V4-3, V3-N3 and V3-5
optionally wherein the repertoire comprises
A antibody heavy chains having cat IGH V gene segments expressed from:
   (i) V3-14, optionally with up to 8 other cat IGH V gene segments;
   (ii) V3-14 and V3-11, optionally with up to 7 other cat IGH V gene segments;
   (iii) V3-14, V3-11 and V3-12, optionally with up to 6 other cat IGH V gene segments;
   (iv) V3-14, V3-11, V3-12, and V3-84, optionally with up to 5 other cat IGH V gene segments;
   (v) V3-14, V3-11, V3-12, V3-84, and V3-20, optionally with up to 4 other cat IGH V gene segments;
   (vi) V3-14, V3-11, V3-12, V3-84, V3-20, and V3-27, optionally with up to 3 other cat IGH V gene segments;
   (vii) V3-14, V3-11, V3-12, V3-84, V3-20, V3-27, and V4-3, optionally with up to 2 other cat IGH V gene segments;
   (viii) V3-14, V3-11, V3-12, V3-84, V3-20, V3-27, V4-3, and V3-N3, optionally with up to 1 other cat IGH V gene segment; or
   (ix) V3-14, V3-11, V3-12, V3-84, V3-20, V3-27, V4-3, V3-N3 and V3-5;
and/or
B antibody heavy chains expressed from IGHV gene segments of cat IGHV families 3 and 4.

Families of genes, such as IGHV family 3, are well-known terms in the art. In an aspect, reference to families of genes, such as IGHV family 3, refers to the notation of the IMGT Repertoire (ImMunoGeneTics), which is available (as of 26 May 2022) at: https://www.imgt.org/IMGTrepertoire/LocusGenes/#F.

An antibody heavy chain repertoire, the repertoire comprising antibody heavy chains having cat IGHV gene segments from no more than 9 different cat IGH V gene segments, where 1 IGHV segment must be IGHV4-3, and at least one of the remaining IGH V segments is selected from the list comprising V3-14, V3-11, V3-12, V3-84, V3-20, V3-27, V4-3, V3-N3 and V3-5.

An antibody lambda light chain repertoire, the repertoire comprising antibody light chains having cat lambda V gene segments from no more than 11 different lambda V gene segments, wherein at least one of the V gene segments is selected from the list comprising V1-124, V1-36, V1-104, V1-33, V1-56, V1-42, V1-41, V3-7, V3-5, V3-13 and V1-126,
optionally wherein the repertoire comprises
A antibody lambda light chains having cat lambda V gene segments including
   (i) V1-124, optionally with up to 10 other cat lambda V gene segments;
   (ii) V1-124 and V1-36, optionally with up to 9 other cat lambda V gene segments;
   (iii) V1-124, V1-36, and V1-104, optionally with up to 8 other cat lambda V gene segments;
   (iv) V1-124, V1-36, V1-104, and V1-33, optionally with up to 7 other cat lambda V gene segments;
   (v) V1-124, V1-36, V1-104, V1-33, and V1-56, optionally with up to 6 other cat lambda V gene segments;
   (vi) V1-124, V1-36, V1-104, V1-33, V1-56, V1-42, optionally with up to 5 other cat lambda V gene segments; or
   (vii) V1-124, V1-36, V1-104, V1-33, V1-56, V1-42, and V1-41, optionally with up to 4 other cat lambda V gene segments;
and/or
B antibody lambda light chains expressed from lambda V gene segments of cat lambda families 1 and 3.

An antibody kappa light chain repertoire, the repertoire comprising antibody kappa light chains having cat kappa V gene segments from no more than 6 different cat kappa V gene segments, wherein at least one of the V gene segments is selected from the list comprising V4-1, V2-12, V2-5, V2-13, V2-9 and V2-4,
optionally wherein the repertoire comprises
A antibody kappa light chains having cat kappa V gene segments including
   (i) V4-1, optionally with up to 5 other cat kappa V gene segments;
   (ii) V4-1 and V2-12, optionally with up to 4 other cat kappa V gene segments;
   (iii) V4-1, V2-12, and V2-5, optionally with up to 3 other cat kappa V gene segments;
   (iv) V4-1, V2-12, V2-5, and V2-13, optionally with up to 2 other cat kappa V gene segments;
   (v) V4-1, V2-12, V2-5, V2-13, and V2-9, optionally with up to 1 other cat kappa V gene segment; or
   (vi) V4-1, V2-12, V2-5, V2-13, V2-4 and V2-4; or
   (vii) V4-1, V2-12, V2-5, V2-13, V2-9 and V2-4;
and/or
B antibody kappa light chains expressed from kappa V gene segments of cat kappa V families 2 and 4.

An antibody kappa light chain repertoire, the repertoire comprising antibody kappa light chains having cat kappa V gene segments from no more than 6 different cat kappa V gene segments, wherein one of the cat kappa V gene segments must be IGKV4-1, and at least one of the V gene segments is selected from the list comprising V4-1, V2-12, V2-5, V2-13, V2-9 and V2-4.

An isolated antibody or antibody chain, or part thereof, or a nucleic acid such as DNA encoding an antibody chain or a part thereof, which has been obtained or is obtainable from a rodent or rodent cell described herein, or from a repertoire described herein, such as a fully cat antibody chain or fully cat antibody, or a pharmaceutical composition comprising the same.

An antibody or antibody or chain, or part thereof, or a nucleic acid such as DNA encoding an antibody chain or a part thereof, which has been obtained or is obtainable from a rodent or rodent cell as described herein, or from a repertoire described herein, such as a fully cat antibody chain or fully cat antibody, or a pharmaceutical composition comprising the same, for use in treatment or prevention of disease in a cat in need thereof.

A method of treatment of a cat, the method comprising delivery of an antibody or antibody chain or part thereof to a cat in need thereof, the antibody being obtained or obtainable from a rodent or rodent cell or repertoire as disclosed herein.

A rodent or rodent cell having a genome comprising a cat IGH V gene segment selected from any one of the sequences set out in Table 1. A rodent or rodent cell having a genome comprising a cat IGH V gene segment selected from the list comprising IGH V3-14 (SEQ ID NO: 1), V1-36 (SEQ ID NO: 11), V1-104 (SEQ ID NO: 12), and V1-42 (SEQ ID NO: 15), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGK V gene segment selected from any one of the sequences set out in Table 1. A rodent or rodent cell having a genome comprising a cat IGK V gene segment IGK V2-5 (SEQ ID NO: 23), and one or more cat IGH J region gene segment(s),
wherein the rodent or rodent cell is capable of expressing the cat gene segments to form an antibody light chain.

### Tables

Table 1—List of preferred V gene sequences
Table 2a—Analysis of healthy cat PBMC, listing the most commonly observed heavy and light chain pairings
Table 2b—Analysis of healthy cat PBMC, listing the most commonly observed heavy chains
Table 2c—Analysis of healthy cat PBMC, listing the most commonly observed light chains
Table 3—Primer sequences
Table 4—Analysis of healthy cat PBMC, listing the light chains ranked by number of different heavy chain pairings
Table 5a—Analysis of healthy cat PBMC, listing the most commonly observed heavy chains
Table 5b—Analysis of healthy cat PBMC, listing the most commonly observed lambda chains
Table 5c—Analysis of healthy cat PBMC, listing the most commonly observed kappa chains

### DETAILED DESCRIPTION

The present invention relates to rodents which comprise cat immunoglobulin gene segments. Prior art rodents, both those containing human IG DNA and cat IG DNA, generally look to insert as many V gene segments as possible to optimise the range of antibodies produced. However, this requires a considerable amount of genome engineering effort to achieve a complete insertion of the large IG locus. In the present disclosure we have analysed the profile of antibodies made in European short hair cats and shown that only a few V gene segments contribute disproportionately to the antibody pool. For example, the cat IGH V3-14 gene segment is found in over 14% of heavy chains of antibodies. Therefore, it is not necessary to insert all of the V gene segments in order to create an antibody population that will provide a suitable repertoire for the identification and selection of lead antibody candidates. This streamlines the genetic engineering needed to make a rodent model for antibody production and enables the production of a rodent model with IGHV and IG light chain V usage which reflects the usage in naive cats.

If a large portion (such as all) of the cat IG locus is inserted into the rodent, some V gene segments preferentially used in cat might be less frequently expressed in the rodent, due to their distance upstream from the (rodent) constant region. This could reduce the effectiveness of such a rodent to produce therapeutically relevant antibodies. Therefore, another advantage of the present invention is that preferred V gene segments can be inserted closer to the constant region than their natural position in cat, and therefore these preferred V gene segments are likely to be used more often in the rodent than if a larger portion of the cat IG locus is inserted, thus more faithfully representing the natural repertoire of a cat.

The cat V gene segments chosen to be inserted into a rodent in order to create an antibody population could be based on observed frequency, on the number of different pairings (between heavy cat V gene segments and light cat V gene segments), or a combination of the two. The highest observed frequency cat V gene segments are preferred. Preferred heavy or light cat V gene segments are those comprised within antibody chains which pair with the highest number of different light or heavy cat V antibody chains respectively (antibody chains that comprise different cat V gene segments).

The invention therefore relates to:

A rodent or rodent cell having a genome comprising;
i) no more than 9 cat IGHV gene segments, one or more cat D region gene segments and one or more cat J region gene segments,
ii) wherein the rodent or rodent cell is capable of expressing the cat gene segments to form an antibody heavy chain, and
iii) wherein at least one of the cat IGH V gene segments is selected from the list comprising V3-14, V3-11, V3-12, V3-84, V3-20, V3-27, V4-3, V3-N3 and V3-5.

Table 2b lists the frequency of observed cat heavy chains V gene segments. One or more of the 9 preferred IGH V gene segments may be combined in the rodent genome with any other cat V gene segment or segments. It is not necessary to limit only to the preferred 9 cat V gene segments, but at least one must be present. A preferred cat gene segment to be present is IGH V3-14. An alternative cat gene segment that is preferred is V3-11. These 2 sequences together are together found in over 23% of the cat antibody heavy chains.

A further embodiment disclosed herein is a rodent or rodent cell disclosed herein comprising cat IGH
(i) V3-14, optionally with up to 8 other cat IGH V gene segments;
(ii) V3-14 and V3-11, optionally with up to 7 other cat IGH V gene segments;
(iii) V3-14, V3-11 and V3-12, optionally with up to 6 other cat IGH V gene segments;
(iv) V3-14, V3-11, V3-12, and V3-84, optionally with up to 5 other cat IGH V gene segments;
(v) V3-14, V3-11, V3-12, V3-84, and V3-20, optionally with up to 4 other cat IGH V gene segments;
(vi) V3-14, V3-11, V3-12, V3-84, V3-20, and V3-27, optionally with up to 3 other cat IGH V gene segments;
(vii) V3-14, V3-11, V3-12, V3-84, V3-20, V3-27, and V4-3, optionally with up to 2 other cat IGH V gene segments;
(viii) V3-14, V3-11, V3-12, V3-84, V3-20, V3-27, V4-3, and V3-N3, optionally with up to 1 other cat IGH V gene segment; or
(ix) V3-14, V3-11, V3-12, V3-84, V3-20, V3-27, V4-3, V3-N3 and V3-5.

A further embodiment disclosed herein is a rodent or rodent cell comprising no more than 8, no more than 7, no more than 6, no more than 5, no more than 4, no more than 3, no more than two or only 1 of the cat IGH V gene segment(s) V3-14, V3-11, V3-12, V3-84, V3-20, V3-27, V4-3, V3-N3 and V3-5.

A further embodiment disclosed herein is a rodent or rodent cell having only 1 cat IGH V gene segment as listed above, and only 1 cat IGH D gene segment and only 1 cat IGH J gene segment, forming a “common heavy chain”.

In an aspect, a common heavy chain approach can be used to generate bispecific antibodies.

A further embodiment disclosed herein is a rodent or rodent cell comprising at least 2 IgH gene segments, such as at least 3, at least 4, at least 5, at least 6, at least 7, at least 8 or all 9 of the cat IGH V gene segments V3-14, V3-11, V3-12, V3-84, V3-20, V3-27, V4-3, V3-N3 and V3-5. The rodent or rodent cell may comprise 1, 2, 3, 4, 5, 6, 7, 8 or 9 of the cat IGH V gene segments V3-14, V3-11, V3-12, V3-84, V3-20, V3-27, V4-3, V3-N3 and V3-5.

In one embodiment of any aspect of the invention, the total number of cat V gene segments inserted at each locus (heavy, lambda and/or kappa) is 9 or fewer.

In one embodiment of any aspect of the invention, the total number of cat V gene segments inserted at each locus (heavy, lambda and/or kappa) is 11 or fewer.

In one embodiment of any aspect of the invention, the total number of cat V gene segments inserted at each locus (heavy, lambda and/or kappa) is 6 or fewer.

In one embodiment of any aspect of the invention, the total number of cat V gene segments inserted at each locus (heavy, lambda and/or kappa) is 20 or fewer.

In one embodiment of any aspect of the invention, the total number of cat V gene segments inserted at each locus (heavy, lambda and/or kappa) is 35 or fewer.

In one embodiment of any aspect of the invention, the total number of cat V gene segments inserted at each locus (heavy, lambda and/or kappa) is 50 or fewer.

It is preferred that the rodent comprises at least one gene segment from each of cat IGH V gene families 3 and 4, there being no more than 9 cat IGHV gene segments in the rodent or rodent cell genome.

In an embodiment, the inserted V genes are all functional V genes.

In an embodiment, the inserted V genes are comprised of functional V genes and non-functional V genes (including pseudogenes and open reading frames).

In an embodiment, a subset of the total number of preferred cat V gene segments is inserted. 1, 2, 3, 4, 5, 6, 7, 8 or 9 of the preferred cat IGH V gene segments may be inserted. 1, 2, 3, 4, 5, 6, 7, 8, 9, 10, or 11 of the preferred cat IGL V gene segments may be inserted. 1, 2, 3, 4, 5 or 6 of the preferred cat IGK V gene segments may be inserted.

The total number of cat V gene segments inserted at each locus may be limited, as disclosed herein, so that in combination, for example, the invention contemplates insertion of 20 cat IGH V gene segments wherein 6 of the inserted cat IGH V gene segments are preferred cat IGH V gene segments.

In a further embodiment, the inserted cat V gene segments are inserted together with other cat V gene segments, and/or other regions of the cat IG locus. In particular, the chromosomal regions surrounding the preferred V gene may be inserted together therewith.

For example, an inserted cat V gene segment as part of a single bacterial artificial chromosome (BAC). In an aspect, each BAC may contain 3-5 cat V gene segments, including one of the preferred cat V gene segments.

In an aspect, reference to a BAC, such as BAC FCAB-223I3 is a reference to the FCAB library from Amplicon Express, now available via College of Veterinary Medicine and Biomedical Sciences, Texas A&M University.

In an aspect, inserting a cat IGH V gene segment may involve insertion of some or all of BAC FCAB-223I3.

In an aspect, inserting a cat IGH V gene segment may involve insertion of some or all of BAC FCAB-253K11. In an aspect, inserting a cat IGH V gene segment may involve insertion of some or all of BAC FCAB-253K11 and some or all of BAC FCAB-180D24. In an aspect, inserting a cat IGH V gene segment may involve insertion of some or all of BAC FCAB-253K11, some or all of BAC FCAB-180D24 and some or all of BAC FCAB-74C2.

In an aspect, inserting a cat IGL V gene segment may involve insertion of some or all of BAC FCAB-3517.

In an aspect, inserting a cat IGL V gene segment may involve insertion of some or all of BAC FCAB-166L22. In an aspect, inserting a cat IGL V gene segment may involve insertion of some or all of BAC FCAB-166L22 and some or all of BAC FCAB-87D23. In an aspect, inserting a cat IGL V gene segment may involve insertion of some or all of BAC FCAB-166L22, some or all of BAC FCAB-87D23 and some or all of BAC FCAB-526E12.

In an aspect, inserting a cat IGK V gene segment may involve insertion of some or all of BAC FCAB-609P16.

In an embodiment, the inserted cat gene segments may be inserted as part of an array or cluster of cat gene segments, optionally embedded in rodent or cat noncoding regulatory or scaffold sequences.

In an embodiment, the inserted cat gene segments may be inserted as part of a minilocus.

Alternative methods of inserting gene segments into a rodent are described in US20170306352, which is incorporated by reference, in the context of a dog and are equally applicable for a cat.

In an embodiment, the inserted cat V gene segments are inserted as part of a concatemer. In an aspect, the concatemer is a series of preferred cat V gene segments, optionally with cat or rodent regulatory sequences associated with the cat V gene segments.

The invention further relates to:

An isolated nucleotide molecule comprising or consisting of the sequence of any of SEQ ID NOs: 1, 11, 12, 15 or 23, or a sequence with at least 80%, 85%, 90%, 95%, 96%, 97%, 98%, or 99% identity thereto, such as at least 95% identity.

A polypeptide comprising or consisting of an amino acid sequence encoded by a nucleotide molecule in accordance with the invention. In one embodiment said polypeptide may be an antibody.

A pharmaceutical composition comprising an antibody in accordance with the invention.

A rodent or rodent cell having a genome comprising the sequence of any of SEQ ID NOs: 1, 11, 12, 15 or 23. Suitable such rodent cell may include an isolated rodent B cell or a cell line such as an ES cell or iPS cell. Mouse ES cell lines are described, for example, in WO2018/1899520.

A vector comprising the sequence of any of SEQ ID NOs: 1, 11, 12, 15 or 23.

A rodent or rodent cell having a genome comprising a cat IGH V gene segment selected from the list comprising IGH V3-14 (SEQ ID NO: 1), V1-36 (SEQ ID NO: 11), V1-104 (SEQ ID NO: 12), and V1-42 (SEQ ID NO: 15), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGH V gene segment IGH V3-14 (SEQ ID NO: 1), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGH V gene segment IGH V3-14 (SEQ ID NO: 1) and a cat IGH V gene segment IGH V1-36 (SEQ ID NO: 11), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGH V gene segment IGH V3-14 (SEQ ID NO: 1), a cat IGH V gene segment IGH V1-36 (SEQ ID NO: 11) and a cat IGH V gene segment IGH V1-104 (SEQ ID NO: 12), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGH V gene segment IGH V3-14 (SEQ ID NO: 1), a cat IGH V gene segment IGH V1-36 (SEQ ID NO: 11), a cat IGH V gene segment IGH V1-104 (SEQ ID NO: 12), and a cat IGH V gene segment IGH V1-42 (SEQ ID NO: 15), one or more cat IGH D region gene segment(s) and one or more cat IGH J region gene segment(s),
wherein the rodent or rodent cell is capable of expressing the cat gene segments to form an antibody heavy chain.

A rodent or rodent cell having a genome comprising a cat IGK V gene segment IGK V2-5 (SEQ ID NO: 23), and one or more cat IGH J region gene segment(s),
wherein the rodent or rodent cell is capable of expressing the cat gene segments to form an antibody light chain.

The invention further relates to:

A rodent or rodent cell having a genome comprising:
i) no more than 11 cat lambda V gene segments, and one or more cat J region gene segments;
ii) wherein the rodent or rodent cell is capable of expressing the cat lambda gene segments to form an antibody light chain,
iii) wherein at least one of the cat lambda gene segments is selected from V1-124, V1-36, V1-104, V1-33, V1-56, V1-42, V1-41, V3-7, V3-5, V3-13 and V1-126.

Table 2c lists the frequency of observed cat light chain V gene segments. One or more of the 11 preferred cat lambda gene segments may be combined in the rodent genome with any other cat V gene segment or segments. It is not necessary to limit only to the preferred 11 cat V gene segments, but at least one must be present. A preferred cat gene segment to be present is IGH V-124. An alternative cat gene segment that is preferred is V1-36. An alternative cat gene segment that is preferred is V1-104. An alternative cat gene segment that is preferred is V1-33. These 4 sequences together are together found in over 17% of the lambda antibody chains.

A further embodiment disclosed herein is a rodent or rodent cell comprising cat lambda V
(i) V1-124, optionally with up to 10 other cat lambda V gene segments;
(ii) V1-124 and V1-36, optionally with up to 9 other cat lambda V gene segments;
(iii) V1-124, V1-36, and V1-104, optionally with up to 8 other cat lambda V gene segments;
(iv) V1-124, V1-36, V1-104, and V1-33, optionally with up to 7 other cat lambda V gene segments;
(v) V1-124, V1-36, V1-104, V1-33, and V1-56, optionally with up to 6 other cat lambda V gene segments;
(vi) V1-124, V1-36, V1-104, V1-33, V1-56, V1-42, optionally with up to 5 other cat lambda V gene segments; or
(vii) V1-124, V1-36, V1-104, V1-33, V1-56, V1-42, and V1-41, optionally with up to 4 other cat lambda V gene segments.

A further embodiment disclosed herein is a rodent or rodent cell comprising no more than 10, no more than 9, no more than 8, no more than 7, no more than 6, no more than 5, no more than 4, no more than 3, no more than 2 or only 1 V gene segment selected from the list of V1-124, V1-36, V1-104, V1-33, V1-56, V1-42, V1-41, V3-7, V3-5, V3-13 and V1-126.

A further embodiment disclosed herein is a rodent or rodent cell having only 1 cat lambda V gene segment, as listed above, and only 1 cat lambda J gene segment, forming a “common light chain”.

A further embodiment disclosed herein is a rodent or rodent cell comprising at least 2 cat lambda V gene segments, such as at least 3, at least 4, at least 5, at least 6, at least 7, at least 8, at least 9, or at least 10 cat lambda V gene segments selected from V1-124, V1-36, V1-104, V1-33, V1-56, V1-42, V1-41, V3-7, V3-5, V3-13 and V1-126. The rodent or rodent cell may comprise 1, 2, 3, 4, 5, 6, 7, 8, 9, 10 or 11 of the cat lambda gene segments V1-124, V1-36, V1-104, V1-33, V1-56, V1-42, V1-41, V3-7, V3-5, V3-13 and V1-126.

It is preferred that the rodent comprises at least one lambda V gene segment from each of the cat lambda V gene segment families 1 and 3, there being no more than 11 cat lambda V gene segments in the genome.

The invention further relates to:

A rodent or rodent cell having a genome comprising:
i) no more than 6 cat kappa V gene segments and one or more cat J region gene segment(s);
ii) wherein the rodent or rodent cell is capable of expressing the cat kappa gene segments to form an antibody light chain,
iii) wherein at least one of the cat kappa gene segments is selected from V4-1, V2-12, V2-5, V2-13, V2-9 and V2-4.

Table 2c lists the frequency of observed cat light chain V gene segments. One or more of the 6 preferred cat kappa gene segments may be combined in the rodent genome with any other cat V gene segments. It is not necessary to limit only to the preferred 6 cat kappa V gene segments, but at least one must be present. A preferred cat gene segment to be present is V4-1, which is seen in over 27% of all kappa chain antibodies. An alternative cat gene segment that is preferred is V2-12. These 2 sequences together are together found in over 50% of the lambda antibody chains.

A further embodiment disclosed herein is a rodent or rodent cell comprising cat kappa V
(i) V4-1, optionally with up to 5 other cat kappa V gene segments;
(ii) V4-1 and V2-12, optionally with up to 4 other cat kappa V gene segments;
(iii) V4-1, V2-12, and V2-5, optionally with up to 3 other cat kappa V gene segments;
(iv) V4-1, V2-12, V2-5, and V2-13, optionally with up to 2 other cat kappa V gene segments;
(v) V4-1, V2-12, V2-5, V2-13, and V2-9, optionally with up to 1 other cat kappa V gene segment; or
(vi) V4-1, V2-12, V2-5, V2-13, V2-4 and V2-4; or
(vii) V4-1, V2-12, V2-5, V2-13, V2-9 and V2-4.

A further embodiment disclosed herein is a rodent or rodent cell comprising no more than 5, no more than 4, no more than 3, no more than 2, or only 1 cat kappa V gene segment selected from V4-1, V2-12, V2-5, V2-13, V2-9 and V2-4.

A further embodiment disclosed herein is a rodent or rodent cell having only 1 cat kappa V gene segment, as listed above, and only 1 cat kappa J gene segment, forming a “common light chain”.

A further embodiment disclosed herein is a rodent or rodent cell comprising at least 2 cat kappa V gene segments, such as at least 3, at least 4, at least 5, or 6 cat kappa V gene segments selected from V4-1, V2-12, V2-5, V2-13, V2-9 and V2-4.

A further embodiment disclosed herein is a rodent or rodent cell comprising 1, 2, 3, 4, 5 or 6 cat kappa V gene segments selected from V4-1, V2-12, V2-5, V2-13, V2-9 and V2-4.

It is preferred that the rodent comprises a kappa V gene segment from each of the kappa V gene segment families 2 and 4, suitably including IGKV2-12 and IGKV4-1, with there being no more than 6 cat kappa V gene segments in the genome.

In another preferred aspect the rodent genome comprises cat DNA encoding any one, or more, of the following heavy and light chain pairs, which together form over 7% of the observed antibody chain pairings:

| | |
|---|---|
| IGHV3-14fc | IGLV1-104fc |
| IGHV3-14fc | IGLV1-124fc |
| IGHV3-14fc | IGLV1-36fc |
| IGHV3-14fc | IGLV1-56fc |
| IGHV3-14fc | IGLV1-126fc |
| IGHV3-14fc | IGLV1-42fc |
| IGHV3-14fc | IGLV1-41fc |
| IGHV3-14fc | IGKV4-1fc |
| IGHV3-14fc | IGLV3-7fc |
| IGHV3-14fc | IGKV2-12fc |
| IGHV3-12fc | IGLV1-104fc |
| IGHV3-11fc | IGLV1-56fc |
| IGHV3-14fc | IGLV1-33fc |
| IGHV3-11fc | IGLV1-41fc |

The inserted DNA in which the number of V gene segments is limited may be referred to herein as the constrained insertion or the constrained locus. It will be appreciated that some embodiments of the invention allow for an unconstrained inserted number of heavy chain V gene segments (when the light chain is constrained), or vice versa allow for an unconstrained inserted number of light chain V gene segments when the heavy chain is constrained. 1, 2 or all 3 cat insertions may be constrained.

The number of cat V gene segments at the constrained locus suitably provides at least 80% coverage of the observed antibody sequences, such as at least 85%, 90%, 95%, 97%, 98%, or 99% coverage, optionally wherein coverage of the observed antibody sequences is less than 100%.

The number of cat V gene segments at the constrained locus, which may be a single cat V gene segment, suitably provides at least 10% coverage of the observed antibody sequences, such as at least 20%, 30%, 40%, 50%, 60% or 70% coverage, optionally wherein coverage of the observed antibody sequences is less than 100%.

The number of cat V gene segments at the constrained locus suitably provides at most 80% coverage of the observed antibody sequences, such as at most 85%, 90%, 95%, 97%, 98%, or 99% coverage.

The number of cat V gene segments at the constrained locus, which may be a single cat V gene segment, suitably provides around 11% coverage of the observed antibody sequences, or around 20%, around 30%, around 40%, around 50%, around 60% or around 70% coverage.

For example, the rodent or cell genome may comprise the constrained cat heavy chain insertion as disclosed herein in combination with a constrained cat lambda light chain insertion as disclosed herein, and/or with a constrained cat light chain kappa chain insertion as disclosed herein.

The rodent or rodent cell genome of the invention may comprise one or more cat IGHV gene segments as described herein, as a constrained insertion, but comprise no light chain cat DNA, or may comprise one or more cat IG light chain V gene segments as described herein as a constrained insertion but no heavy chain cat DNA.

Where an immunoglobulin locus of a rodent or rodent cell genome is not constrained by the number of V gene segments, it may have any number, including all, of the cat V gene segments. The rodent or cell genome may therefore comprise an inserted cat heavy chain as disclosed herein in combination with any light chain insertion—such as a fully cat light chain, or a cat light chain insertion having more than 11 cat V lambda gene segments and/or more than 6 cat kappa V gene segments. Alternatively, the rodent or cell genome may comprise the cat light chain insertion as disclosed herein in combination with any heavy chain—such as a fully cat heavy chain, or a cat heavy chain insertion having more than 9 cat V gene segments.

For example, where only the heavy chain insertion is constrained by the number of V gene segments, the light chain locus may have one, or more or all of the light cat V gene segments. It may comprise at least 50% of the cat light chain variable (V) genes for kappa and/or lambda, such as at least 60%, at least 70%, at least 80%, at least 90%, and in one aspect all of the cat kappa and/or lambda V genes.

In one embodiment the rodent or rodent cell genome may comprise at least 5, 10, 20, 30, 40, 50, 60, 70, 80, 90, or at least 100 cat IG lambda V region genes, optionally functional cat IG lambda V region genes. In one preferred embodiment the rodent genome comprises at least 78 cat IG light chain lambda V region genes, optionally functional cat IG light chain lambda V region genes.

In one embodiment the rodent or rodent cell genome may comprise at least 5, 6, 7, 8, 9 or at least 10 cat IG kappa V region genes, optionally functional cat IG kappa V region genes. In one preferred embodiment the rodent genome comprises at least 10 cat IG light chain kappa V region genes, optionally functional cat IG light chain kappa V region genes.

For example, where only the light chain insertion is constrained, the heavy chain locus may have one, or more or all of the heavy chain cat IGH V gene segments. It may comprise at least 50% of the cat heavy chain variable (V) genes, such as at least 60%, at least 70%, at least 80%, at least 90%, and in one aspect all of the cat heavy chain V genes. In this aspect the rodent or rodent cell genome may comprise at least 4, 5, 10, 15 or 20, cat IGH V region genes, such as at least 20, 30, 40, or at least 50 cat IGH V region genes, optionally functional cat IGH V region genes. In one preferred aspect the rodent genome comprises at least 34 cat IG heavy chain V region genes, optionally functional cat IG heavy chain V region genes.

References herein to genes may, where appropriate, be references to gene segments. Use of the term ‘gene’ is not intended to exclude such features being equivalently disclosed in relation to a gene segment, unless where otherwise dictated by context or necessity.

Similarly, references herein to gene segments may, where appropriate, be references to genes. Use of the term ‘gene segment’ is not intended to exclude such features being equivalently disclosed in relation to a gene, unless where otherwise dictated by context or necessity.

The preferred cat DNA is European short hair cat DNA, and therefore preferred cells and rodents comprise European short hair cat DNA gene segments, as disclosed herein. The use of European short hair cat regulatory sequences are also preferred. The European short hair cat DNA may be provided as genomic DNA.

In all embodiments and aspects of the invention the cat gene segments may be located in the rodent genome upstream of a rodent constant region, suitably upstream of the heavy chain constant region for inserted cat heavy chain variable region gene segments and suitably upstream of a light chain constant region for inserted cat light chain variable region gene segments, such that the rodent or rodent cell is able to produce a chimaeric antibody heavy chain, or chimaeric light chain, or both, resulting from expression of the inserted variable region gene segments and a host constant region.

Any reference to the location of the variable region upstream of a constant region, such as the rodent constant region, means that there is a suitable relative location of the two genomic portions, encoding the variable and constant regions of the antibody, to allow a chimaeric antibody chain to be expressed in vivo in the rodent. In this way the inserted cat DNA and constant region are in functional arrangement with one another for antibody or antibody chain production.

Information concerning, or the nucleic acid comprising, the variable region of a chimaeric antibody chain may be obtained from the cells expressing chimaeric antibodies using standard techniques. These sequences can be used to generate fully cat antibodies by expression of the nucleic acid encoding the antibody variable region with a cat constant region to generate a cat antibody, for therapeutic use in cats for example.

The cat DNA may be inserted at the rodent wild-type constant region located at the wild type locus, suitably between the rodent constant region and the host VDJ or VJ region. The rodent constant region expressed with the cat variable region is preferably the rodent wild-type constant region located at the wild type locus, as appropriate for the cat heavy or light chain VDJ or VJ. In one aspect the IGH variable region genes are inserted downstream of the rodent heavy chain J region, and upstream of the Emu enhancer.

In one aspect the IGH variable region genes are inserted downstream of the rodent heavy chain J region, and upstream of the Emu enhancer. In one aspect the rodent is a mouse and insertion of IGH V region genes is made at position 114666435 of the mouse genome, on mouse chromosome 12. In one aspect the insertion of IG lambda V region genes is made at position 19047551 of the mouse genome, on chromosome 16. In one aspect the insertion of IG kappa V region gene or genes is made at position 70674755 of the mouse genome, on chromosome 6.

Preferably the inserted cat V(D)J gene segments can undergo V(D)J rearrangement to form an antibody chain in the rodent. However, where a single rearranged heavy or light chain variable region is inserted, or a complete antibody chain VDJC or VJC is inserted, no gene segment rearrangement may be needed.

In a preferred embodiment, the rodent (or rodent cell) of the invention is a mouse (or mouse cell). In an alternative, the inserted cat gene segments are located (inserted) into the genome in functional arrangement with a cat constant region, such that the rodent is able to produce an antibody chain resulting from the expression of the inserted cat VDJ gene segments with a cat constant region, and/or an antibody chain resulting from the expression of the inserted cat VJ gene segments with a cat constant region.

One possibility is the expression of a fully cat antibody light chain with a chimaeric heavy chain having a cat VDJ and rodent heavy chain constant region. One possibility is the expression of a fully cat antibody heavy chain with a chimaeric light chain having a cat VJ and rodent heavy chain constant region.

References herein such as ‘the inserted cat gene segments’ and ‘the inserted cat DNA’ refer to the cat sequences which are present in the genome of the rodent or rodent cell, and therefore have been inserted in the genome of the rodent or rodent cell.

In one aspect the inserted cat DNA comprises at least 50% of the cat heavy chain diversity (D) genes, such as at least 60%, at least 70%, at least 80%, at least 90%, and in one aspect all of the cat D genes.

In one aspect the inserted cat DNA comprises at least 50% of the cat heavy chain joining (J) genes, such as at least 60%, at least 70%, at least 80%, at least 90%, and in one aspect all of the cat J genes.

In one aspect the inserted cat DNA comprises at least 50% of the cat light chain joining (J) genes, such as at least 60%, at least 70%, at least 80%, at least 90%, and in one aspect all of the cat light chain J genes.

The rodent or rodent cell genome may comprise at least 1, 2, 3, 4, 5, 6, 7, 8, 9, 10, or 11 IGHD region gene segments from a cat.

The rodent or rodent cell genome may comprise at least 1, 2, 3, 4, or 5 IGHJ region genes from a cat.

The rodent or rodent cell genome may comprise at least 1, 2, 3, 4, or 5 IG kappa J region genes from a cat.

The rodent or rodent cell genome may comprise at least 1, 2, 3, 4, 5, 6, 7, 8, 9, 10, 11, 12, 13, 14, 15, 16, 17, or 18 IG lambda J region genes from a cat.

The number of cat genes referred to above in any aspect may also be further increased, and in one aspect is doubled, in the case of a homozygote having an insertion at both alleles.

One possible insertion of cat V gene segments as described above is upstream of the rodent constant region to generate a chimaeric antibody chain expressed at the endogenous locus.

In other aspects the inserted cat DNA is located in the rodent genome at a site which is distinct from that of the naturally occurring heavy or light constant region, such as on a different chromosome. The insertion may be at a random location into the rodent genome. In this case the insertion of the VDJ or VJ region genes is accompanied by a constant region, and preferably also by a 3′ enhancer, from the rodent or from the cat.

One preferred embodiment is the use of a rodent constant region and rodent 3′ enhancer with cat VDJ or VJ regions, or the use of a cat constant region and cat 3′ enhancer with cat VDJ or VJ regions. In one aspect the cat gene segments(s) are located in the genome in functional arrangement with the constant region such that the rodent is able to produce an antibody chain.

The invention also specifically contemplates cells and rodents having an insertion of cat genes in association with a cat constant region (encoding a “fully” cat antibody chain) at an endogenous rodent IG locus, such as DNA encoding a cat lambda V and J and C genes.

In one aspect the fully cat VJC lambda antibody chain described above is inserted into the rodent lambda locus, preferably between the last rodent C gene and 3′ enhancer.

In one aspect the rodent or rodent cell comprises one or more cat IG lambda V region genes, one or more cat IG lambda J region genes and one or more cat lambda constant region genes located at the rodent cell kappa locus, for example within the locus or upstream or downstream of the rodent kappa constant region. Preferably the insertion is upstream of the IG kappa locus constant region, such that the IG lambda V and J region genes are expressed with the IG kappa constant region.

Suitably the insertion locates the cat genes at approximately the same position as the native rodent kappa genes, potentially deleting or displacing them, for example there being the same or substantially the same distance from the last inserted 3′ lambda J gene to the rodent kappa constant region gene as there is from the last rodent kappa 3′ J gene to the kappa constant region.

In one aspect the insertion is within 1, 2, 3, 4, 5, 6, 7, 8, 9, or 10 kb of the boundary (upstream or downstream) of the rodent immunoglobulin kappa locus. The rodent kappa light chain is naturally expressed at a higher level than the rodent lambda light chain, and insertion of the cat DNA at this kappa locus can provide high levels of expression of the cat lambda chain V region genes.

In one aspect the cat lambda V and/or J gene segments are associated with cat regulatory or scaffold sequences. In another aspect the cat lambda V and/or J gene segments are associated with regulatory or scaffold sequences from a rodent, such as the regulatory or scaffold sequences native to the rodent cell used for insertion of the cat DNA.

In one aspect where cat lambda V genes are inserted at the rodent kappa locus, the rodent kappa locus, or part thereof, may be deleted or rendered non-functional, for example by mutation. Inactivation or rendering an endogenous mouse or rodent K light chain locus nonfunctional to increase expression of lambda light chain is described, for example, in WO2021003149, in the context of a dog and is equally applicable for a cat.

References herein to gene segments being associated with regulatory or scaffold sequences, and similar such statements, refer to gene segments operably linked to the regulatory or scaffold sequences. The term ‘associated’ in this context means ‘operably linked’ and may be interchangeable therewith.

In one aspect the rodent genome is homozygous for the inserted cat genes. Such homozygous rodents are preferred for use in the generation of cat antibodies by immunization.

The insertion may be at the native rodent IG loci. In another aspect the rodent genome may be heterozygous for the insertion of cat genes at one, or two, or three loci, such as immunoglobulin loci.

The rodent genome may be heterozygous for the insertion of cat genes at the kappa locus.

Where the inserted cat DNA at the constrained IgH locus comprises at least 4 IGH variable region genes, preferably one of those is V3-14.

In one aspect the rodent genome is modified to reduce or prevent expression of fully rodent antibodies that have both variable and constant regions from the rodent. This may be by inversion of all or part of the rodent VDJ region and/or VJ region, or by a deletion of all or part of the rodent VDJ region and/or VJ region, or by an insertion into the endogenous rodent VDJ and/or VJ region of the genome. For example, an insertion of cat DNA at an IG locus upstream of the rodent constant region will move the host rodent V (D) and J gene segments further away from the rodent constant region and reduce expression or inactivate the host rodent antibody expression from that locus.

In one aspect the rodent VDJ or VJ region or a part thereof is deleted.

In one aspect all or a number of the rodent V region genes are deleted, such as:
at least 50%, preferably at least 75% or at least 90%, or all, of the rodent IGH V gene segments and/or D gene segments and/or J gene segments, and/or
at least 50% preferably at least 75% or at least 90%, or all, of the rodent IG light chain V gene segments and/or J gene segments from kappa and/or lambda.

In one aspect the rodent is a mouse and the genome comprises a deletion of one or some or all of the mouse IGH V region genes, preferably from V1-85 to V5-2.

In one aspect the rodent is a mouse and the genome comprises a deletion of one or some or all of the mouse IG kappa V region genes, preferably from V3-1 to V2-137.

In one aspect the rodent is a mouse and the mouse heavy chain D and J region genes are retained in the genome upstream of the inserted cat heavy chain variable region genes.

In one aspect the rodent IG lambda genes are not deleted from the rodent genome.

In one aspect one or both alleles of the rodent kappa locus are deleted or inactivated, in whole or in part, by insertion of cat DNA at the rodent kappa locus.

In one aspect the rodent kappa locus is inactivated wholly or partially, for example, by insertion, or by deletion or by inversion.

In one aspect the rodent lambda locus is inactivated, wholly or partially, for example, by insertion, or by deletion or by inversion.

In one aspect the rodent heavy chain locus is inactivated, wholly or partially, for example, by insertion, or by deletion or by inversion.

The cat variable region gene segments(s) are suitably inserted upstream of rodent constant region, the latter comprising all of the DNA required to encode the full constant region or a sufficient portion of the constant region to allow the formation of an effective chimaeric antibody capable of specifically recognising an antigen.

Reference to a chimaeric antibody or antibody chain having a rodent constant region herein therefore is not limited an antibody chain having the complete constant region but also includes chimaeric antibodies or chains which have a part of the constant region, sufficient to provide one or more effector functions seen in antibodies occurring naturally in the rodent. Effector functions include the ability to interact with Fc receptors, and/or bind to complement. Likewise, the cat variable region DNA may be located in the host genome such that it forms a chimaeric antibody chain with all or part of a rodent constant region to form an antibody chain or a part thereof respectively.

Preferably the rodent genome comprises all of the cat heavy constant region DNA and intervening regions. Preferably the rodent genome comprises all of the cat heavy constant region genes and intervening regions.

Preferably the rodent genome comprises all of the cat lambda constant region DNA and intervening regions. Preferably the rodent genome comprises all of the cat lambda constant region genes and intervening regions.

Preferably the rodent genome comprises all of the cat kappa constant region DNA and intervening regions. Preferably the rodent genome comprises all of the cat kappa constant region genes and intervening regions.

In one aspect the inserted cat DNA is capable of being expressed with different rodent constant regions through isotype switching.

In one aspect the inserted cat DNA is capable of being expressed with a different rodent constant region through trans-switching.

In one embodiment one, or more, or all of the inserted cat V, D or J region gene segments is associated with a cat regulatory sequence, preferably from the same cat breed.

In one aspect at least one rodent enhancer or other control sequence, such as a switch region, is maintained in functional arrangement with the rodent constant region. In this way the effect of the enhancer or other control sequence may be exerted in whole or in part in the cell or transgenic rodent.

In one aspect one or more rodent control sequences, such as the Emu enhancer sequence, is maintained upstream of the rodent Mu constant region, suitably in its native position with respect to the distance from the constant region.

In one aspect one or more rodent control sequences such as an enhancer sequence(s) is/are maintained downstream of the rodent constant region, suitably in its native position with respect to the distance from the constant region.

In one aspect the rodent Smu switch sequence is maintained upstream of the rodent Mu constant region, suitably in its native position with respect to distance from the constant region. In such location the rodent enhancer or switch sequence is suitably operative in vivo with the host constant region sequence(s).

In a further aspect one or more of the promoter elements, or other control elements, of the cat V, D or J region genes is/are optimised in the genome to interact with the transcriptional machinery of the rodent.

In one aspect the rodent or rodent cell genome comprises one or more cat promoters, or enhancers, and/or other control elements associated with the cat V, D or J gene segments.

In one aspect the one or more cat control regions (elements), such as promoters or enhancers or switch regions, replace one or more rodent promoters or enhancers or switch regions respectively. The cat control sequences are suitably maintained in functional arrangement with a constant region such that the effect of the control sequence(s) may be exerted in whole or in part in the cell or transgenic rodent.

In one aspect at least one or more of the inserted cat V, D or J gene segments is associated with a regulatory sequence, such as a recombination signal sequence (RSS), from the same cat, optionally wherein the regulatory sequences direct the successful recombination of the V, D or J gene segment or segments.

In one aspect, the ‘same’ cat refers to the same breed of cat. In one aspect, the same cat refers to precisely the same animal.

In one aspect, at least one or more of the inserted cat V, D or J gene segments is directly associated cis or trans with a regulatory sequence, or flanked on one or both sides with a regulatory sequence, optionally wherein the one or more gene segments is directly flanked by the regulatory sequence.

In one aspect the regulatory sequences comprise a promoter preceding an individual V gene segment, and/or a splice site within an individual V gene segment, and/or a recombination signal sequences for V(D)J recombination downstream of a V gene segment, flanking a D gene segment or upstream of a J gene segment.

In one aspect a V, D or J sequence of the inserted cat DNA is flanked by an RSS sequence from the same cat. For example, a cat RSS sequence may be used with cat V, D and/or J sequences. It will be appreciated that this can be provided by insertion of a genomic fragment from the cat into the rodent genome.

In a further aspect, the invention provides a method of replacing, in whole or part, in a rodent cell an endogenous immunoglobulin variable region gene locus with a cat gene locus comprising: obtaining a cloned genomic fragment or a synthetic sequence containing, in whole or in part, the companion gene locus comprising at least one V, or D (for the heavy chain) or J gene segments, and at least one associated regulatory sequence, and insertion of the cat DNA into the genome of a rodent, suitably at an endogenous rodent immunoglobulin locus, preferably the heavy or light chain rodent locus corresponding to the nature of the inserted cat DNA.

In one aspect the inserted cat DNA is associated with rodent regulatory sequences which allow for V(D)J recombination in the rodent. Such an approach is disclosed, for example, in US20170306352, which is incorporated by reference, in the context of a dog and is equally applicable for a cat.

In one aspect the regulatory sequences are non-coding regulatory sequences which comprise the following sequences of endogenous host origin: promoters preceding each V gene segment coding sequence, introns, splice sites, and recombination signal sequences for V(D)J recombination. In another aspect the regulatory sequence is one or more of: promoters preceding each V gene segment coding sequence, introns, splice sites, and recombination signal sequences for V(D)J recombination, all of which are of endogenous host origin.

In another aspect a partly feline immunoglobulin locus generated in the invention comprises one or more of the following sequences of endogenous host origin: ADAM6A or ADAM6B gene, a Pax-5-Activated Intergenic Repeat (PAIR) elements, or CTCF binding sites from a heavy chain intergenic control region 1.

In one aspect the invention relates to a transgenic rodent, such as a mouse, with a genome in which an entire endogenous immunoglobulin variable gene locus has been deleted and replaced with an engineered partly feline immunoglobulin locus comprising cat immunoglobulin variable gene V H, D and J H and/or cat V L and J L coding sequences as described herein, and rodent immunoglobulin variable gene locus non-coding regulatory sequences, wherein the engineered partly feline immunoglobulin locus of the transgenic rodent is functional and expresses immunoglobulin chains comprised of feline variable domains and rodent constant domains.

The rodent may be a mouse as described in US20170306352, the disclosure of which is incorporated by reference.

The rodent may be a mouse as described in WO2021003149 the disclosure of which is incorporated by reference.

In one aspect the inserted cat DNA at the constrained locus is not a genomic DNA fragment. In order to insert the gene segment or segments with the highest natural representation, the inserted DNA may comprise DNA in which the gene segments are not in their natural germline configuration and in genomic DNA. The inserted cat DNA may be a minigene having multiple different V gene segments, for example.

Inserted cat V gene segments at the constrained locus may be arranged with gene segment or gene segments of most interest located closest to the constant region. For example, the IGHV 3-14 gene segment may be the most closely located to the constant region to maximise expression. Gene segments located closer to the constant region are usually expressed at higher levels. Alternatively, the gene segments with the highest natural occurrence in the cat antibody population may be located further from the constant region, to provide a balanced repertoire.

The present invention provides a rodent or rodent cell which expresses a limited number of preferred V gene segments, such as one gene segment, and includes as a preferred feature the presence of only a single V, D and J gene segment for the heavy chain variable region, and/or a single V and J for the light chain variable region in the genome. In this way the rodent will generate only a single type of variable region for the heavy chain, and/or a single type of variable region for the light chain of the cat antibody chain that is produced.

In an aspect, the term ‘single type of variable region’ refers to a variable region with only one V, (one D,) and one J gene segment sequence.

Such an arrangement can provide a common light chain, useful in the generation of bispecific antibodies, where a single light chain is desired, and is expressed with 2 different heavy chains in the bispecific antibody.

Therefore, the invention also relates to a method for the generation of a bispecific antibody, the method comprising immunizing a rodent as disclosed herein with an antigen, the rodent comprising only a single type of cat light chain variable region (single cat V and J gene segments, cat VJ) in the rodent genome, and selecting for a bispecific antibody capable of binding to that antigen, and suitably also to a second preferred antigen target.

The invention also relates to a bispecific antibody obtained or obtainable from a method of the invention, wherein the antibody light chain is obtained from expression of a preferred V gene segment as disclosed herein, suitably from a rodent of the invention. Suitably the bispecific has a preferred light and heavy chain pairing as disclosed herein.

Any bispecific antibody format may be used, such as any of those disclosed in Brinkmann U and Kontermann RE, MAbs. 2017 February-March; 9 (2): 182-212.

The cat V gene segment chosen to be used in the common light chain may be based on frequency (Table 2b and Table 2c), number of different heavy chain cat V gene segment pairings (Table 4), or a combination of both.

In another aspect, this disclosure explicitly contemplates use of IGHV3-16 (SEQ ID NO: 45) instead of IGHV3-N3 (SEQ ID NO: 8). References herein to IGHV3-N3 or SEQ ID NO: 8 can equally be considered to be references to IGHV3-16 or SEQ ID NO: 45 for the purposes of this aspect of the invention.

In another aspect, this disclosure explicitly contemplates use of IGLV1-N1 (SEQ ID NO: 46) instead of IGLV1-33 (SEQ ID NO: 13). References herein to IGLV1-33 or SEQ ID NO: 13 can equally be considered to be references to IGHV1-N1 or SEQ ID NO: 46 for the purposes of this aspect of the invention.

In another aspect, this disclosure explicitly contemplates use of IGLV1-N14 (SEQ ID NO: 47) instead of IGLV1-41 (SEQ ID NO: 16). References herein to IGLV1-41 or SEQ ID NO: 16 can equally be considered to be references to IGHV1-N14 or SEQ ID NO: 47 for the purposes of this aspect of the invention.

This disclosure explicitly also contemplates use of IGHV3-16 (SEQ ID NO: 45) instead of IGHV3-N3 (SEQ ID NO: 8), use of IGLV1-N1 (SEQ ID NO: 46) instead of IGLV1-33 (SEQ ID NO: 13) and use of IGLV1-N14 (SEQ ID NO: 47) instead of IGLV1-41 (SEQ ID NO: 16).

The term “bispecific antibody” means an antibody which comprises specificity for two target molecules, and includes formats such as DVD-Ig (see DiGiammarino et al., “Design and generation of DVD-Ig™ molecules for dual-specific targeting”, Meth. Mo. Biol., 2012, 889, 145 156), mAb2 (see WO2008/003103), FIT-Ig (see WO2015/103072), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. For a review of bispecific formats, see Spiess, C., et al., Mol. Immunol. (2015).

In another embodiment, the bispecific molecule comprises an antibody which is fused to another non-Ig format, for example a T-cell receptor binding domain: an immunoglobulin superfamily domain; an agnathan variable lymphocyte receptor: a fibronectin domain (e.g., an Adnectin™): an antibody constant domain (e.g., a CH3 domain, e.g., a CH2 and/or CH3 of an Fcab™) wherein the constant domain is not a functional CH1 domain; an scFv; an (scFv) 2; an sc-diabody; an scFab; a centyrin and an epitope binding domain derived from a scaffold selected from CTLA-4 (Evibody™); a lipocalin domain; Protein A such as Z-domain of Protein A (e.g., an Affibody™ or SpA); an A-domain (e.g., an Avimer™ or Maxibody™); a heat shock protein (such as and epitope binding domain derived from GroEI and GroES); a transferrin domain (e.g., a trans-body); ankyrin repeat protein (e.g., a DARPin™); peptide aptamer; C-type lectin domain (e.g., Tetranectin™); human γ-crystallin or human ubiquitin (an affilin); a PDZ domain; scorpion toxin; and a kunitz type domain of a human protease inhibitor.

Antibody structure has been exploited to engineer a variety of different antibody formats to target disease in humans. An example of such an engineered antibody format is bispecific antibodies. Bispecific antibodies bind to two different targets and are therefore capable of simultaneously binding to two different epitopes. One area of interest is T cell directed bispecific antibodies for efficient tumour killing. Bispecific antibodies can have “two-target” functionality and bind to two different surface receptor or ligands thus influencing multiple disease pathways.

Bispecific antibodies can also place two targets in close proximity, either to support protein complex formation on one cell or to trigger contact between cells. Bispecific antibodies formats vary in many ways including their molecular weight, number of antigen-binding sites, spatial relationship between different binding sites, valency for each antigen, ability to support secondary immune functions and pharmacokinetic half-life. These diverse formats provide great opportunity to tailor the design of bispecific antibodies to match the proposed mechanisms of action and the intended clinical application (Kontermann and Brinkmann Bispecific Antibodies Drug Discovery Today Volume 20, Number 7, 2015).

Production of bispecific antibodies of the IgG type by co-expression of the two light and two heavy chains in a single host cell can be highly challenging because of the low yield of desired bispecific IgGs and the difficulty in removing closely related mispaired IgG contaminants. This reflects that heavy chains form homodimers as well as the desired heterodimers—the so-called heavy chain-pairing problem. Additionally, light chains can mispair with non-cognate heavy chains—the so-called light chain pairing problem. Consequently, coexpression of two antibodies can give rise to up to nine unwanted IgG species in addition to the desired bispecific antibody.

Various approaches are described in the art in order to promote heterodimerisation, i.e. the formation of a certain bispecific antibody of interest for human therapy, thereby reducing the content of undesired homodimers in the resulting mixture.

In one embodiment, the antibody is a multispecific antibody or fragment thereof. A multispecific protein, e.g. a multispecific antibody, binds to at least two different targets, i.e. is at least bispecific. Thus, in one embodiment, the antibody is a bispecific antibody or fragment thereof. In other embodiment, the multispecific antibody or fragment thereof binds to three, four or more targets.

A bispecific antibody has specificity for no more than two epitopes. A bispecific antibody is characterised by a first immunoglobulin variable domain sequence which has binding specificity for a first epitope and a second immunoglobulin variable domain sequence that has binding specificity for a second epitope.

In one embodiment, the first and second epitopes are on the same antigen, e.g., the same protein (or subunit of a multimeric protein). In one embodiment, the first and second epitopes overlap. In an embodiment, the first and second epitopes do not overlap. In one embodiment, the first and second epitopes are on different antigens, e.g., the different proteins (or different subunits of a multimeric protein).

In another embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In a further embodiment, a bispecific antibody molecule comprises an antibody having binding specificity for a first epitope and an antibody having binding specificity for a second epitope. In one embodiment, a bispecific antibody molecule comprises an antibody, or fragment thereof, having binding specificity for a first epitope and a half antibody, or fragment thereof, having binding specificity for a second epitope. In one embodiment, a bispecific antibody or fragment thereof comprises a Fab having binding specificity for a first epitope and a Fab having binding specificity for a second epitope.

Bispecific antibodies of the invention based on the IgG format, comprising of two heavy and two light chains can be produced by a variety of methods known in the art. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers), which contain two identical paired heavy chains with the same specificity, and bispecific dimers (also called heterodimers) which contain two different paired heavy chains with different specificity. In addition, light chains and heavy chains from each antibody may randomly pair to form inappropriate, non-functional combinations. This problem, known as heavy and light chain miss-pairings, can be solved by choosing antibodies that share a common light chain for expression as bispecifics. Methods to address the light chain-heavy chain mispairing problem include the generation of bispecific antibodies using a single light chain. This requires heavy-light chain engineering or novel antibody libraries that utilise a single light chain that limits the diversity. In addition, antibodies with a common light chain have been identified from transgenic mice with a single light chain. Another approach is to swap the CH1 domain of one heavy chain with CL domain of its cognate light chain (Crossmab technology). Also covered are scFv formats.

Methods for making bispecific antibodies are described in WO2019008123 and WO2014160179, which are incorporated by reference.

The invention also relates to a method for the generation of a bispecific antibody, the method comprising immunizing a first rodent as disclosed herein with a first antigen, the rodent comprising only a single cat lambda light chain variable region (single cat V and J gene segments, cat VJ) in the rodent genome, and selecting for an antibody capable of binding to said first antigen, and immunizing a second rodent as disclosed herein with a second antigen, the rodent comprising the same single cat lambda light chain variable region (single cat V and J gene segments, cat VJ) in the rodent genome, and selecting for an antibody capable of binding to said second antigen, optionally wherein the single cat lambda light chain V gene segment is selected from the list comprising cat lambda V1-124, V3-7, V1-42, V1-104, V1-56, V1-126, V3-13, V3-5, V3-36, V1-41 and V1-33.

In one aspect, the single lambda cat light chain V gene segment is V1-124.

In one aspect, the single lambda cat light chain V gene segment is V3-7.

The invention also relates to a bispecific antibody obtained or obtainable from a method of the invention, wherein the cat antibody light chain is obtained from expression of a single preferred lambda V gene segment as disclosed herein, suitably from a rodent of the invention. Suitably the bispecific has a preferred light and heavy chain pairing as disclosed herein. In one aspect, the single lambda cat V gene segment is selected from the list comprising cat lambda V1-124, V3-7, V1-42, V1-104, V1-56, V1-126, V3-13, V3-5, V3-36, V1-41 and V1-33.

The present invention provides a rodent or rodent cell which includes a single cat lambda V and J for the light chain variable region in the genome. In this way the rodent will generate only a single type of variable region for the light chain of the cat antibody chain that is produced. In one aspect, the single cat lambda light chain V gene segment is V1-124. In one aspect, the single lambda cat light chain V gene segment is V3-7.

The invention also relates to a method for the generation of a bispecific antibody, the method comprising immunizing a first rodent as disclosed herein with a first antigen, the rodent comprising only a single cat kappa light chain variable region (single cat V and J gene segments, cat VJ) in the rodent genome, and selecting for an antibody capable of binding to said first antigen, and immunizing a second rodent as disclosed herein with a second antigen, the rodent comprising the same single cat kappa light chain variable region (single cat V and J gene segments, cat VJ) in the rodent genome, and selecting for an antibody capable of binding to said second antigen, optionally wherein the single cat kappa light chain V gene segment is selected from the list comprising cat kappa V2-5, V2-9, V2-12, V2-13, V4-1 and V2-4.

In one aspect, the single kappa cat light chain V gene segment is V4-1.

In one aspect, the single kappa cat light chain V gene segment is V2-5.

The invention also relates to a bispecific antibody obtained or obtainable from a method of the invention, wherein the cat antibody light chain is obtained from expression of a single preferred kappa V gene segment as disclosed herein, suitably from a rodent of the invention. Suitably the bispecific has a preferred light and heavy chain pairing as disclosed herein. In one aspect, the single kappa cat V gene segment is selected from the list comprising cat kappa V2-5, V2-9, V2-12, V2-13, V4-1 and V2-4.

The present invention provides a rodent or rodent cell which includes a single cat kappa V and J for the light chain variable region in the genome. In this way the rodent will generate only a single type of variable region for the light chain of the cat antibody chain that is produced. In one aspect, the single cat kappa light chain V gene segment is V4-1. In one aspect, the single lambda cat light chain V gene segment is V2-5.

The invention also relates to a bispecific antibody wherein both light chains are identical and are selected from the list comprising cat lambda V1-124, V3-7, V1-42, V1-104, V1-56, V1-126, V3-13, V3-5, V3-36, V1-41 and V1-33.

In an embodiment, both light chains are cat lambda V1-124.

In an embodiment, both light chains are cat lambda V3-7.

The invention also relates to a bispecific antibody wherein both light chains are identical and are selected from the list comprising cat kappa V2-5, V2-9, V2-12, V2-13, V4-1 and V2-4.

In an embodiment, both light chains are cat lambda V4-1.

In an embodiment, both light chains are cat lambda V2-5.

In one aspect the rodent cell of the invention is a rodent ES cell, rodent hematopoietic stem cell or other cell capable of developing into a rodent able to produce a repertoire of antibody chains comprising a variable region encoded by cat DNA, such as chimaeric antibody heavy chains or chimaeric antibody light chains, or fully cat antibody chains or antibodies encoded by cat variable regions having variable and constant regions.

In one aspect the cell of the invention is a rodent ES cell or an induced pluripotent stem cell (iPS cell). Such cells are suitable for the insertion of cat DNA to generate rodents expressing antibody chains as described herein.

In one aspect the cell is an isolated rodent cell.

In one aspect the cell is an isolated rodent B cell.

The rodent or rodent cell of the invention is preferably a mouse or rat, or mouse or rat cell (such as a mouse or rat ES cell), and is preferably a mouse or mouse ES cell. The ES cell may be of mouse cell strain 129 or C57BL, such as strain C57BL/6N, C57BL/6J, 129S5 or 129Sv strains, or in a cell which has a hybrid genome which comprises 129 and/or C57BL genomic DNA.

The invention also relates to a cell line which is grown from or otherwise derived from cells as described herein, including an immortalised cell line.

The cell or cell line of the invention may comprise cat V, (D) or J genes in unrearranged configuration or after rearrangement following in vivo maturation.

The invention also relates to a cell or cell line, such as a CHO cell line, expressing an antibody chain having a variable region which is obtainable by immunising a rodent of the invention with an antigen, or where the nucleic acid sequence of the variable region may be identified from, or has been identified from a rodent or rodent cell as described herein, or from a repertoire of antibodies described herein. The antibody chain may be a chimaeric antibody heavy chain or is preferably a fully cat antibody chain. The cell or cell line expressing the antibody chain or antibody may be a CHO cell, or other mammalian cell line suitable for the production of a therapeutic for animal use. The cell may be immortalised by fusion to a tumour cell to provide an antibody producing cell and cell line, or be made by direct cellular immortalisation.

The present invention also relates to vectors for use in the invention. In one aspect such vectors are bacterial artificial chromosomes (BACs) comprising all or a part of the cat IG locus suitable for insertion into an ES cell. It will be appreciated that other cloning vectors may be used in the invention, and therefore reference to BACs herein may be taken to refer generally to any suitable vector. The vector may comprise one or more selectable markers and/or one or more site specific recombination sites. In one aspect the vector comprises 2 or more, such as 3, heterospecific and incompatible site-specific recombination sites. In one aspect the site-specific recombination sites may be loxP sites, or variants thereof, or FRT sites or variants thereof. In one aspect the vector comprises one or more transposon ITR (inverted terminal repeat) sequences.

Suitable BACs containing cat DNA are available as the FCAB library from Amplicon Express, now available via College of Veterinary Medicine and Biomedical Sciences, Texas A&M University.

In one aspect some or all of the inserted DNA is from a European short hair cat.

Preferably the cell or rodent comprises cat V, D and J gene segments from the FCAB BAC library from Amplicon Express, now available via College of Veterinary Medicine and Biomedical Sciences, Texas A&M University.

In one aspect one or more cat gene segment alleles used in the present invention are the reference alleles for each cat gene. In an aspect, these are those of FelisCatus8.0—see assembly accession—GCA_000181335.3, assembled November 2014. In one aspect, these are those of FelisCatus9.0—see assembly accession—GCF_000181335.3, assembled November 2017.

In one aspect at least 90%, at least 95% and preferably all of the cat gene segments that have been inserted are the cat reference alleles from CanFam 3.1. In one aspect, at least 50, 60, 70, 80, 90 or 100% of the inserted V gene segments are cat V gene reference alleles, and/or at least 50, 60, 70, 80, 90 or 100% of the inserted D gene segments are cat D gene reference alleles, and/or at least 50, 60, 70, 80, 90 or 100% of the inserted J gene segments are cat J gene reference alleles, and combinations of these.

In one aspect, references to cat gene segments (such as IGHV3-14) are references to the equivalent gene segments of FelisCatus8.0 or FelisCatus9.0. However, it will be understood that such sequences can be different from the exact sequence of FelisCatus8.0 or FelisCatus9.0, such as 99% identical, 98% identical, 97% identical, 96% identical or 95% identical.

In one aspect, reference to cat gene segments (such as IGHV3-14) are references to gene segments having the relevant sequence in Table 1.

In one aspect, reference to cat gene segments (such as IGHV3-14) are references to gene segments having the relevant sequence in Table 1 or having a sequence identity of 80%, 90%, 95%, 96%, 97%, 98%, or 99% thereto.

In one aspect, the rodent or rodent cell comprises one or more reference alleles of the cat gene segments.

Preferably the V (D) and J region gene segments that are inserted into the genome are all from the same breed of cat or the same cat.

In one aspect the rodent is able to generate a diversity of at least 1×10⁶ different functional chimaeric immunoglobulin sequence combinations.

It is noted that reference herein to ‘cat’, or reference herein to ‘feline’, are intended to be interchangeable, and both terms refer to Felis catus.

The invention also relates to methods for making rodents comprising inserted cat DNA, methods for making antibodies and antibody chains from those rodents, and methods for making pharmaceutical compositions comprising cat antibody chains or antibodies.

A method for producing a rodent or rodent cell disclosed herein, the method comprising inserting into a rodent cell genome
(i) no more than 9 cat IGHV gene segments, one or more cat IGH D region gene segments and one or more cat IGH J region gene segments, wherein at least one of the cat IGH V gene segments is selected from the list comprising V3-14, V3-11, V3-12, V3-84, V3-20, V3-27, V4-3, V3-N3 and V3-5; and/or
(ii) no more than 11 cat lambda V gene segments and one or more cat lambda J region gene segments, wherein at least one of the cat IGH V gene segments is selected from the list comprising V1-124, V1-36, V1-104, V1-33, V1-56, V1-42, V1-41, V3-7, V3-5, V3-13 and V1-126; and/or
(iii) no more than 6 cat kappa V gene segments and one or more cat J region gene segments, wherein at least one of the cat kappa gene segments is selected from V4-1, V2-12, V2-5, V2-13, V2-9 and V2-4;
and wherein the rodent or rodent cell is capable of expressing the cat variable region gene segment or segments in combination with a constant region to form an antibody chain.

Methods for targeted insertion of exogenous DNA at the endogenous rodent locus are well known in the art, such that inserted V, D and J genes can be expressed with the host constant region. The same principles can be applied to insertion of feline DNA. See Murphy et al, Vol 111 no 14, 5153-5158, doi: 10.1073/pnas.1324022111; MacDonald et al vol. 111 no. 14, 5147-5152, doi: 10.1073/pnas. 1323896111; and Lee et al, Nature Biotechnology Volume: 32, Pages: 356-363 Year published:, 2014 DOI:, doi: 10.1038/nbt.2825. Suitable methods for the construction of a chimaeric IG locus encoding feline VDJ and or VJ gene segments with a constant region are disclosed in WO2018189520, the methods of which are incorporated herein by reference.

Preferably the method relates to inserting both light chain and heavy chain cat VDJ and VJ region genes respectively such that an antibody is produced in which both light and heavy chains have a variable region derived from expression of cat DNA.

In one aspect rodent ES cells carrying one or more chimeric loci are used to make chimaeras in which the host embryos are generated from a RAG-1-deficient background, or other suitable genetic background which prevents the production of mature host B and T lymphocytes. This enables all the B and T cells to be derived from the injected ES cells.

The ES cells of the present invention can be used to generate animals using techniques well known in the art, which may for example comprise injection of the ES cell into a blastocyst followed by implantation of chimaeric blastocysts into females to produce offspring, which can be bred to produce heterozygous offspring which are then interbred to produce homozygous recombinants having the required insertion. In one aspect the host blastocysts are Rag-deficient, such as RAG-1-deficient.

The invention relates to a chimaeric rodent generated by injection of an ES cell of the invention into a blastocyst followed by implantation of chimaeric blastocysts into a rodent female to produce offspring.

In one preferred aspect the rodent or rodent cell is a mouse or mouse cell and the mouse ADAM6a and ADAM6b genes are present in the mouse genome, and have not been previously deleted then reinserted from the IGH locus.

In one aspect the rodent ADAM6a and ADAM6b genes are located at a position 5′ of the inserted one or more cat V, D and J genes.

In one aspect the rodent IGH D and J genes are present in the rodent genome. In one aspect the rodent IGH D and J genes have not been deleted from the rodent genome. In one aspect the rodent IGH D and J genes are located at a position 5′ of the inserted one or more cat V, D and J gene segments.

The invention also relates to a method for producing an antibody or antibody chain specific to a desired antigen, the method comprising immunizing a rodent as disclosed herein with the desired antigen and recovering the antibody or antibody chain, singularly or as part of a complete antibody, or recovering a cell producing the antibody or antibody chain, singularly or as part of a complete antibody (see e.g. Harlow, E. & Lane, D. 1998, 5^{th} edition, Antibodies: A Laboratory Manual, Cold Spring Harbor Lab. Press, Plainview, NY; and Pasqualini and Arap, Proceedings of the National Academy of Sciences (2004) 101:257-259).

Suitably an immunogenic amount of the antigen is delivered. The invention also relates to a method for detecting a target antigen comprising detecting an antibody produced as above with a secondary detection agent which recognises a portion of that antibody.

The invention also relates to a method for producing an antibody chain or antibody specific to a desired antigen the method comprising immunizing a rodent comprising cat gene segments located upstream of a constant region which is not from a cat, as disclosed herein, and then replacing the constant region of the antibody chain or antibody with that of a cat constant region, suitably by engineering of the nucleic acid encoding the antibody. Preferably the constant region is from the same cat breed. Standard cloning techniques are known to replace the non-human mammal constant region with an appropriate cat constant region DNA sequence—see e.g. Sambrook, J and Russell, D. (2001, 3'd edition) Molecular Cloning: A Laboratory Manual (Cold Spring Harbor Lab. Press, Plainview, NY). Alternatively direct nucleic acid synthesis may be used to generate the fully cat sequence using sequence information from the DNA encoding the chimaeric antibody.

The invention also relates to a method comprising identifying cat variable regions by single cell sequencing and making a vector to express a full antibody chain with the corresponding cat constant region. The invention also relates to obtaining these cat sequences by PCR and joining them to an appropriate constant region, such as a cat constant region, by a suitable molecular biology technique such as but not limited to bridge-PCR and Gibson cloning. The DNA may also be synthesised for inclusion in an expression vector.

In a yet further aspect, chimaeric antibodies or antibody chains generated in the present invention may be manipulated, suitably at the DNA level, to generate molecules with antibody-like properties or structure, such as:
a cat variable region from a heavy or light chain absent a constant region,
a domain antibody,
a cat variable region with any constant region from either heavy or light chain from the same or different species
a cat variable region with a non-naturally occurring constant region; or a cat variable region together with any other fusion partner. The invention relates to all such chimaeric antibody derivatives derived from chimaeric antibodies identified according to the present invention.

The invention also relates to a method for making an antibody, or part thereof, the method comprising providing:
(i) a nucleic acid encoding an antibody, or a part thereof, obtained or obtainable according to the present invention; or
(ii) sequence information from which a nucleic acid encoding an antibody obtained or obtainable according to the present invention, or part thereof, can be expressed to allow an antibody to be produced; and
expressing the antibody chain.

Also disclosed is a method for producing an antibody chain or part thereof, the antibody chain having a cat variable region, the method comprising expressing in a cell a nucleic acid such as DNA encoding the antibody chain, or a part thereof,
wherein the sequence of the nucleic acid encoding the variable region of the antibody chain is obtained from, or obtainable by immunising a rodent disclosed herein with an antigen so that antibody chains are produced, or obtained from an antibody of a repertoire as described herein,
optionally including the subsequent steps of:
purifying and/or isolating the antibody chain, and
optionally then formulating the antibody into a pharmaceutically acceptable formulation suitable for administration into a cat.

Also disclosed is a method of making a pharmaceutical composition, the method comprising producing an antibody according to a method disclosed herein and further comprising combining the antibody with a pharmaceutically acceptable carrier or other excipient to produce the composition.

The invention further relates to chimaeric antibodies or antibody chains expressed from gene segments identified as important in generating feline antibodies, and nucleic acids encoding the same, such as:

A nucleic acid encoding a chimaeric antibody chain, the nucleic acid having a variable domain which comprises any one of the cat IGHV gene segments selected from the list comprising V3-14, V3-11, V3-12, V3-84, V3-20, V3-27, V4-3, V3-N3 and V3-5 and a rodent constant region, optionally wherein the cat V gene segment is a sequence that has undergone somatic hypermutation in a rodent.

An antibody chain having a variable region obtained by expression of any one of cat IGH V3-14, V3-11, V3-12, V3-84, V3-20, V3-27, V4-3, V3-N3 and V3-5 in a rodent, in conjunction with a rodent constant region.

A nucleic acid encoding a chimaeric antibody chain, the nucleic acid having a variable domain which comprises any one of the cat lambda V gene segments selected from the list comprising lambda V1-124, V1-36, V1-104, V1-33, V1-56, V1-42, V1-41, V3-7, V3-5, V3-13 and V1-126, and a rodent constant region, optionally wherein the cat V gene segment is a sequence that has undergone somatic hypermutation in a rodent.

An antibody chain having a variable region obtained by expression of any one of cat lambda V1-124, V1-36, V1-104, V1-33, V1-56, V1-42, V1-41, V3-7, V3-5, V3-13 and V1-126 in a rodent, in conjunction with a rodent constant region.

A nucleic acid encoding a chimaeric antibody chain, the nucleic acid having a variable domain which comprises any one of the cat kappa V gene segments selected from the list comprising V4-1, V2-12, V2-5, V2-13, V2-9 and V2-4 and a rodent constant region, optionally wherein the cat V gene segment is a sequence that has undergone somatic hypermutation in a rodent.

An antibody chain having a variable region obtained by expression of any one of cat kappa V4-1, V2-12, V2-5, V2-13, V2-9 and V2-4 in a rodent, in conjunction with a rodent constant region.

The antibody, or antibody chain, or nucleic acid may be obtained or be obtainable from a rodent or rodent cell, or be obtained from a repertoire of antibodies as disclosed herein.

The invention also relates to a part of, or a whole, immunoglobulin molecule comprising cat variable domains and rodent constant domains derived from the constrained insertion of a B cell from a rodent, and a hybridoma cell obtainable or obtained from that B cell, and a part of, or whole, immunoglobulin molecule comprising cat variable domains and rodent constant domains obtainable from or obtained from that hybridoma cell.

In another aspect the invention relates to fragments and functional derivatives of said antibodies and chains disclosed herein, also referred to as parts of antibody chains, and use of said antibodies, chains and fragments in medicine, including diagnosis, and in vitro or ex vivo studies. Functional antibody fragments/parts can include a fragment that is capable of specific binding to an antigen.

A functional antibody fragment may be, for example, a FAB or a single chain variable fragment (scFv). The fragment may comprise at least the variable region of the antibody. The fragment may comprise at least the CDR regions. Suitably the parts are functional in that they can bind a desired antigen—preferably the same antigen used to immunise the rodent to stimulate antibody production.

The invention also relates to nucleic acid, such as DNA or RNA, encoding said antibody, antibody chains, or parts thereof. In particular the part may be the variable portion of the antibody chain, which is that part encoded by the cat DNA within the rodent.

In one aspect the antibody or fragment comprises any combination exemplified in the Examples and Tables herein, or any derivative thereof which is a fully cat antibody or a fragment thereof, capable of antigen binding.

Antibodies of the invention may be isolated, in one aspect being isolated from the cell or organism in which they are expressed.

The present invention relates to both polyclonal and monoclonal antibodies of chimaeric or fully cat antibodies, which may be produced in response to antigen challenge in rodents of the present invention, or derived therefrom, as described herein, and/or which may comprise the V gene segments identified herein as being highly utilised, such as IGH V3-14. Methods for the generation of both monoclonal and polyclonal antibodies are well known in the art.

Also disclosed herein are pharmaceutical compositions comprising said antibodies and antibody chains, or nucleic acid encoding the antibodies or chains.

The invention relates to a pharmaceutical composition comprising an antibody chain having a variable region comprising any one of cat IGH V3-14, V3-11, V3-12, V3-84, V3-20, V3-27, V4-3, V3-N3 and V3-5, in conjunction with a pharmaceutically acceptable excipient or carrier.

A pharmaceutical composition comprising an antibody chain having a variable region comprising any one of cat lambda V1-124, V1-36, V1-104, V1-33, V1-56, V1-42, V1-41, V3-7, V3-5, V3-13 and V1-126 in conjunction with a pharmaceutically acceptable excipient or carrier.

A pharmaceutical composition comprising an antibody chain having a variable region comprising any one of cat kappa V4-1, V2-12, V2-5, V2-13, V2-9 and V2-4, in conjunction with a pharmaceutically acceptable excipient or carrier.

A pharmaceutical composition comprising an antibody, the antibody having
(i) a heavy chain having a variable region obtained by expression of any one of cat IGH V3-14, V3-11, V3-12, V3-84, V3-20, V3-27, V4-3, V3-N3 and V3-5, and either
(ii) a lambda light chain obtained by expression of any one of cat lambda V1-124, V1-36, V1-104, V1-33, V1-56, V1-42, V1-41, V3-7, V3-5, V3-13 and V1-126; or
(iii) a kappa light chain obtained by expression of any one of cat kappa V4-1, V2-12, V2-5, V2-13, V2-9 and V2-4;
   the antibody being formulated in conjunction with a pharmaceutically acceptable excipient or carrier.

Suitable excipients and carriers are well known in the art and include water, surfactants, carbohydrates (e.g., cyclodextrin derivatives) and amino acids.

The invention also relates to a pharmaceutical composition comprising a fully cat antibody packaged within a delivery vehicle, such as an IV bag, or injection device.

The antibody chain may be a chimaeric or fully cat antibody chain.

The invention also relates to repertoires of antibodies, however made. For example, the repertoire may be derived from a transgenic rodent such as a mouse or alternatively a synthetic antibody repertoire such as a phage display system utilising the same limited set of immunoglobulin V gene segments as disclosed herein for the inserted heavy and/or light chain cat DNA. Suitable methods for the generation of synthetic antibody repertoires are described for example in WO2018234438, which is incorporated by reference, in the context of a dog and are equally applicable for a cat.

The invention therefore relates to an antibody heavy chain repertoire, the repertoire comprising antibody heavy chains having cat IGHV gene segments from no more than 9 different cat IGH V gene segments, wherein at least one of the cat IGH V gene segments is selected from the list comprising V3-14, V3-11, V3-12, V3-84, V3-20, V3-27, V4-3, V3-N3 and V3-5, optionally wherein at least 2, at least 3, at least 4, at least 5, at least 6, at least 7, at least 8, or all 9 cat IGH V gene segments is selected from the list comprising V3-14, V3-11, V3-12, V3-84, V3-20, V3-27, V4-3, V3-N3 and V3-5.

Optionally the repertoire comprises antibody heavy chains having cat IGHV gene segments expressed from
(i) V3-14, optionally with up to 8 other cat IGH V gene segments;
(ii) V3-14 and V3-11, optionally with up to 7 other cat IGH V gene segments;
(iii) V3-14, V3-11 and V3-12, optionally with up to 6 other cat IGH V gene segments;
(iv) V3-14, V3-11, V3-12, and V3-84, optionally with up to 5 other cat IGH V gene segments;
(v) V3-14, V3-11, V3-12, V3-84, and V3-20, optionally with up to 4 other cat IGH V gene segments;
(vi) V3-14, V3-11, V3-12, V3-84, V3-20, and V3-27, optionally with up to 3 other cat IGH V gene segments;
(vii) V3-14, V3-11, V3-12, V3-84, V3-20, V3-27, and V4-3, optionally with up to 2 other cat IGH V gene segments;
(viii) V3-14, V3-11, V3-12, V3-84, V3-20, V3-27, V4-3, and V3-N3, optionally with up to 1 other cat IGH V gene segment; or
(ix) V3-14, V3-11, V3-12, V3-84, V3-20, V3-27, V4-3, V3-N3 and V3-5.

In one embodiment the repertoire comprises antibody heavy chains expressed from IGHV gene segments of cat IGHV families 3 and 4.

Also disclosed is an antibody light chain repertoire, the repertoire comprising antibody light chains having cat lambda V gene segments from no more than 11 different cat lambda V gene segments, wherein at least one of the cat lambda V gene segments is selected from the list comprising V1-124, V1-36, V1-104, V1-33, V1-56, V1-42, V1-41, V3-7, V3-5, V3-13 and V1-126, optionally wherein at least 2, at least 3, at least 4, at least 5, at least 6, at least 7, at least 8, at least 9, at least 10, or all 11 cat lambda V gene segments is selected from the list comprising V1-124, V1-36, V1-104, V1-33, V1-56, V1-42, V1-41, V3-7, V3-5, V3-13 and V1-126.

Optionally the repertoire comprises antibody lambda light chains having cat lambda V gene segments including
(i) V1-124, optionally with up to 10 other cat lambda V gene segments;
(ii) V1-124 and V1-36, optionally with up to 9 other cat lambda V gene segments;
(iii) V1-124, V1-36, and V1-104, optionally with up to 8 other cat lambda V gene segments;
(iv) V1-124, V1-36, V1-104, and V1-33, optionally with up to 7 other cat lambda V gene segments;
(v) V1-124, V1-36, V1-104, V1-33, and V1-56, optionally with up to 6 other cat lambda V gene segments;
(vi) V1-124, V1-36, V1-104, V1-33, V1-56, V1-42, optionally with up to 5 other cat lambda V gene segments; or
(vii) V1-124, V1-36, V1-104, V1-33, V1-56, V1-42, and V1-41, optionally with up to 4 other cat lambda V gene segments.

In another embodiment the repertoire comprises antibody lambda light chains expressed from lambda V gene segments of cat lambda families 1 and 3.

Also disclosed is an antibody light chain repertoire, the repertoire comprising antibody kappa light chains having cat kappa V gene segments from no more than 6 different cat kappa V gene segments, wherein at least one of the cat kappa V gene segments is selected from the list comprising V4-1, V2-12, V2-5, V2-13, V2-9 and V2-4, optionally wherein at least 2, at least 3, at least 4, at least 5, or all 6 cat kappa V gene segments is selected from the list comprising V4-1, V2-12, V2-5, V2-13, V2-9 and V2-4.

Optionally the repertoire comprises antibody kappa light chains having cat kappa V gene segments including
(i) V4-1, optionally with up to 5 other cat kappa V gene segments;
(ii) V4-1 and V2-12, optionally with up to 4 other cat kappa V gene segments;
(iii) V4-1, V2-12, and V2-5, optionally with up to 3 other cat kappa V gene segments;
(iv) V4-1, V2-12, V2-5, and V2-13, optionally with up to 2 other cat kappa V gene segments;
(v) V4-1, V2-12, V2-5, V2-13, and V2-9, optionally with up to 1 other cat kappa V gene segment; or
(vi) V4-1, V2-12, V2-5, V2-13, V2-4 and V2-4; or
(vii) V4-1, V2-12, V2-5, V2-13, V2-9 and V2-4.

In another embodiment the repertoire comprises antibody kappa light chains expressed from kappa V gene segments of cat kappa V families 2 and 4.

The antibodies and antibody chains disclosed herein may be used in methods of prevention or treatment of disease in a cat. In particular, fully cat antibodies, which have a cat constant region, may be used.

The invention thus relates to an antibody or antibody or chain, or part thereof, as disclosed herein, such as an antibody obtained or obtainable from a rodent or rodent cell disclosed herein, or from a repertoire disclosed herein, such as a fully cat antibody made using the sequence information from a rodent or rodent cell, for use in treatment or prevention of disease a cat in need thereof.

The invention also relates to a method of treatment of a cat, the method comprising delivery of an antibody or antibody chain, or part thereof as disclosed herein, to a cat in need thereof, the antibody being, for example, an antibody obtained or obtainable from a rodent or rodent cell disclosed herein, or from a repertoire as disclosed herein, or a fully cat antibody made using the sequence information from a rodent or rodent cell.

In a further aspect the invention relates to use of a rodent as described herein as a model for the testing of drugs and vaccines. The invention therefore relates to a method for identification or validation of a drug or vaccine, the method comprising delivering the vaccine or drug to a mammal of the invention, such as a cat, and monitoring one or more of: the immune response, the safety profile: the effect on disease.

The invention also relates to a kit comprising an antibody or antibody derivative as disclosed herein and either instructions for use of such antibody or a suitable laboratory reagent, such as a buffer, antibody detection reagent or excipient for formulation with the antibody.

Certain preferred gene segments are disclosed herein, and we have provided the relevant sequences: IGH V gene segments selected from the list comprising V3-14, V3-11, V3-12, V3-84, V3-20, V3-27, V4-3, V3-N3 and V3-5 or from the list comprising V3-14, V3-11, V3-12, V3-84, V3-20, V3-27, V4-3, V3-16 and V3-5.

Lambda gene segments selected from the list comprising V1-124, V1-36, V1-104, V1-33, V1-56, V1-42, V1-41, V3-7, V3-5, V3-13 and V1-126 or from the list comprising V1-124, V1-36, V1-104, V1-N1, V1-56, V1-42, V1-N14, V3-7, V3-5, V3-13 and V1-126.

Kappa gene segments selected from the list comprising V4-1, V2-12, V2-5, V2-13, V2-9 and V2-4.

The invention also relates to the cat D gene segment (IGHD-N1). The cat D gene segment has the following DNA sequence (SEQ ID NO: 48):

GTGACTACTGTATCGGGAGTAGCTGTGCTAC

The invention also relates to a vector or cell genome or cell line genome or rodent genome comprising cat D gene segment IGHD-N1.

This disclosure explicitly contemplates use of IGHD-N1 (SEQ ID NO: 48) in the rodents, rodent cells and methods, antibodies, pharmaceutical compositions, etc., described herein, both in combination with—and separate from—the V gene segments described herein.

For example, in an embodiment, the invention relates to a rodent or rodent cell having a genome comprising:
one or more cat IGHV gene segments, cat D gene segment IGHD-N1, and one or more cat IGH J region gene segment(s),
wherein the rodent or rodent cell is capable of expressing the cat gene segments to form an antibody heavy chain,
optionally wherein there are no more than 9 cat IGHV gene segments and wherein at least one of the cat IGH V gene segments is selected from the list comprising V3-14, V3-11, V3-12, V3-84, V3-20, V3-27, V4-3, V3-16N3 and V3-5.

This disclosure also explicitly contemplates use of IGHD-N1 (SEQ ID NO: 48) in hosts other than rodents. Therefore, reference herein to ‘rodent’ can be read as ‘non-human animal’ (or ‘non-human mammal’) in these contexts.

As for the cat V gene segments disclosed herein, the use of IGHD-N1 can prevent the need to insert all cat D genes into a host in order to create an antibody population that will provide a suitable repertoire for the identification and selection of antibody candidates for therapeutic and prophylactic treatments.

TABLE 1

| V gene | | |
|---|---|---|
| segment | Sequence | SEQ ID NO |
| Heavy chain | | |
| IGHV3-14 | GATGTGCAGCTGGTGGAATCTGGGGGAGACCTGGTGA | 1 |
| | AGCCTGGGGGGTCCCTGAGACTCACCTGTGTGGCCTCT | |
| | GGATTCACCTTCAGTAGCTATAGCATGAACTGGGTCCG | |
| | CCAGGCTCCAGGGAAGGGGCTGCAGTGGGTCGCATAT | |
| | ATTAGTAGTAGTGGAGGTAGCATATACTACGCAGACTC | |
| | CGTGAAGGGCCGATTCACCATCTCCAGAGACAACGCC | |
| | AAGAACACGCTGTATCTGCAGATGAACAGCCTGAAGA | |
| | CCGAGGACACGGCCACATATTACTGTGCGAGTGA | |
| IGHV3-11 | GACGTGCAATTGGTGGAGTCTGGGGGAGACCTGGTGA | 2 |
| | AGCCTGGGGGGTCCCTGAGACTCACCTGTGTGGCCTCT | |
| | GGATTCACCTTCAGTAGCTATGCAATGAGCTGGGTCCG | |
| | CCAGGCTCCAGGGAAGGGGCTGCAGTGGGTCGCAGAT | |
| | ATTAGTAGTAGTGGAGGTAGCACATACTACGCAGACTC | |
| | CGTGAAGGGCCGATTCACCATCTCCAGAGACAATGCC | |
| | AAGAACACGCTGTATCTGCAGATGAACAGCCTCAAGA | |
| | CCGAGGACACGGCCACATATTACTGTGCGAAAGA | |
| IGHV3-12 | GATGTGCAGCTGGCGGAATCTGGGGGAGACCTGGTGA | 3 |
| | AGCCTGGGGGGTCCCTGAGACTCACCTGTGTGGCCTCT | |
| | GGATTCACCTTCAGTAGCTACTACATGCACTGGGTCCG | |
| | CCAGGCTCCAGGGAAGGGGCTGCAGTGGGTCGCACAA | |
| | ATTAGTAGTAGTGGAGGTAGCACATACTACGCAGACTC | |
| | CGTGAAGGGCCGATTCACCATCTCCAGAGACAACGCC | |
| | AAGAACACGCTGTATCTGCAGATGAACAGCCTGAAGA | |
| | CCGAGGACACGGCCACATATTACTGTGCGAGTGA | |
| IGHV3-84 | GATGTGCAGCTGGTGGAATCTGGGGGAGACCTGGTGA | 4 |
| | AGCCTGGGGGGTCCCTGAGACTCACCTGTGTAGCCTCT | |
| | GGATTCACCTTCAGTAGCTATGACATGAACTGGGTCCG | |
| | CCAGGCTCCAGGGAAGGGGCTGCAGTGGGTCGCATAT | |
| | ATTAGTAGTAGTGGAGGTAGCATATACTACGCAGACTC | |
| | CGTGAAGGGCCGATTCACCATCTCCAGAGACAACGCC | |
| | AAGAACACGCTGTATCTGCAGATGAACAGCCTGAAGA | |
| | CCGAGGACACGGCCACATATTACTGTGCGAGTGA | |
| IGHV3-20 | GACGAGCAGCTGGTGGAGTCTGGGGGAGACCTGGTGA | 5 |
| | AGCCTGGGGGGTCCCTGAGACTCACCTGTGTGGCCTCC | |
| | GGATTCAACGTCGGTAGCTATGGAATGAGCTGGTTCCG | |
| | CCAGGCTCCAGGAAAGGGGCTGCAGTGGGTCGCAGCA | |
| | ATTAGTGGTAGTGGAGGTAGCACAGGTTACGCAGACT | |
| | CCGTGAAGGGCCGATTCACCATCTCCATTGACAACGCC | |
| | AAGAACACGCTGTATCTGCAGATGGACAGCCTGAAGA | |
| | CTGAGGACACGGCCACATATTACTGTGCGAGAGG | |
| IGHV3-27 | GACGTGCAGCTGGTGGAGTCTGGGGGAGACCTGGTGA | 6 |
| | AGCCTGGGGGGTCCCTGAGACTCACCTGTGTAGCCTCT | |
| | GGATTCACCTTCAGTAGCTATGACATGAGCTGGGTCCG | |
| | CCAGGCTCCAGGGAAGGGGCTGCAGTGGGTCGCAGCA | |
| | ATTAGTTATAATGGAGGTAGCACAGGCTACGCAGACTC | |
| | CGTGAAGGGCCGATTCACCATCTCCAGAGACAACGCC | |
| | AAGAACACGCTGTATCTGCAGATGAACAGCCTCAAGA | |
| | CCGAGGACACGGCCACATATTACTGTGCGAGAGA | |
| IGHV4-3 | CAACTTACACTTCGGGAGTCCGGCCCAGGACTGGTGAA | 7 |
| | GCCTTCACAATCCCTCTCTCTCACCTGCGTTGTCTCCGG | |
| | AGGCTCTGTTACCAGCAGTTACTACTGGAACTGGATCC | |
| | GCCAGCGCCCTGGGAGAGGGTTGGAGTGGCTGGGGTA | |
| | CTGGTCAGGTAGCACCAGCTACAACCCGGCTTTCCAGG | |
| | GCCGCATCTCCATCACTGCTGACACAGCCCAGAACCAG | |
| | TTCTCCCTGCAGCTGAGCTCCATGACCACCGAGGACAC | |
| | GGCCGTGTATTACTGTGCAAGAAG | |
| IGHV3-N3 | GACGTGCAGCTGGTGGAGTCTGGGGGAGACCTGGTGA | 8 |
| | AGCCTGGGGGGTCCCTGAGACTCACCTGTGTGGCCTCT | |
| | GGATTCACCTTCAGTAGCAATGCAATGAGCTGGGTCCG | |
| | CCAGGCTCCAGGGAAGGGGCTGCAGTGGGTCTCAGCA | |
| | ATTAGTTATGATGGAGGTAGCACAGGCTACGCAGACTC | |
| | CGTGAAGGGCCGATTCACCATCTCCAGAGACAACGCC | |
| | AAGAACACACTGTATCTGCAGATGAACAGCCTCAAGA | |
| | CCGAGGACACGGCCACATATTACTGTGCAAGAGA | |
| IGHV3-5 | GACGTGCAGCTGGTGGAGTCTGGGGGAGACCTGGTGA | 9 |
| | AGCCTGGGGGGTCCCTGAGACTCACCTGTGTGTTGCCT | |
| | CTGGATTCACCTTCAGTAGCTATTGGATTAATCAGTAT | |
| | CTATTGGATGAGCTGGGTCCGCCAGGCTCCAGGAAAG | |
| | GGGCTGCAGTGGGTCGCAGGAATTAGTGGTAGTGGAG | |
| | GTAGCACAGGCTACGCAGACTCCGTGAAGGGCCGATT | |
| | CACTATCTCCATTGACAACGCCAAGAACACGCTGTATC | |
| | TGCAGATGGACAGCCTGAAGACTGAGGACACGGCCAC | |
| | ATATTACTGTGCGAGAGG | |
| IGHV3-16 | ATGGAGATTGTGCTGGGCTGGGTTTTCCTGGTTGCTCTT | 45 |
| | TTAAAAGGTGTCCAGTGTGATGTGCAGTTGGTGGAGTC | |
| | TGGAGGAGATCTGGTGCAGCCTGGGGGGTCCCTGAGA | |
| | CTCACCTGTGTGGCCTCTGGATTCACCTTCAGTAGCTAT | |
| | AGCATGAACTGGGTCCGCCAGGCTCCAGGGAAGGGGC | |
| | TGCAGTGGGTCGCATATATTAGATATGATGGAAGTAGC | |
| | ACAGCCTACGCAGACTCCGTGAAGGGCCGATTCACCAT | |
| | CTCCAGAGACAACGCCAAGAACACGCTGTATCTGCAG | |
| | ATGAACAGCCTCAAGACCGAGGACACGGCCACATATT | |
| | ACTGTGCG | |
| LAMBDA V | | |
| IGLV1-124 | CAGTCGGGGCCGAATCAACCATCCTCAGTGTCTGGGGC | 10 |
| | CCTGGGCCAGAGGGTCACTATCTCCTGCACTGGAGTTG | |
| | GTAGTTACGTGGACTGGTACCAACAAATCCCAGGAAT | |
| | GGCCCCCAAAACCATCATCTATGGTAATAGCTACCGAC | |
| | CCTCAGGGGTCCCTGATCGATTCTCCGGCTCCAAGTCT | |
| | GGCAGCACAGGCACCCTGACCATCACTGGGCTCCAGG | |
| | CCGAAGACGAGGCTGATTATTACTGCTCATCGTGGGAC | |
| | AGCAGTGGCAGAGCTTA | |
| IGLV1-36 | CAGTCTAGGCTGACTCAGCCGCCCTCAGTGTCTGGTTC | 11 |
| | TCTGGGCCAGAGGGTCACCATCTCCTGCGCTGGAAGCA | |
| | GCTCTAATATTGGTGGTTATGGTGTGAACTGGCACCAA | |
| | CAATTCCCAGGAATGGCCCCCAAAACCATCATCTATGG | |
| | TAATAGCAATCGACCCTCTGGGGTCCCAGATCGATTCT | |
| | CCGGCTCCAAGTCTGGCAACACAGGCACCCTGACCATC | |
| | ACTGGGCTCCAGGCTGAGGACGAGGCTGATTATTACTG | |
| | CTCATCGTGGGACAGCAGTAGCAGTGCTCG | |
| IGLV1-104 | CAGTCGGGGCCGAATCAACCATCCTCAGTGTCTGGGGC | 12 |
| | CCTGGGCCAGAGGGTCACTATCTCCTGCACTGGAATTG | |
| | ATAGTTACGTGGGTTGGTACCAACAAATCCCAGGAATG | |
| | GCCCCCAAAACCATCATCTATGGTAATAGCAACCGACC | |
| | CTCAGGGGTCCCTGATCGATTCTCCGGCTCCAAGTCTG | |
| | GCAGCACAGGCACTCTGACCATCACTGGGCTCCAGGCC | |
| | GAGGACGAGGCTGATTATTACTGCGTATCGTGGTACAG | |
| | CAGTGGCAGAGCTTA | |
| IGLV1-33 | CAGTCTGTGCTGACTCAGCCGCCCTCAGTGTCTGGGGC | 13 |
| | CCTGGGCCAGACGGTCACCATCTCCTGCGCTGGAAGTG | |
| | CCAACAACATTGGTATTGTTGGTGTGAACTGGTACCAG | |
| | CAGCTTCCAGGAAAGGCCCCTAACTCCTCATCTATGCT | |
| | AATAACAGACGACCCTCAAGTGTTCCTGACAGATTTTC | |
| | TGGCTCCAAGTCTGGCAACACAGGCTCCTTGACCATCA | |
| | CTGGGCTCCAGGCTGAGGACGAGGCTGATTATTACTGC | |
| | CTGTCTGCTGACGTTACTCAAGGTGCTGA | |
| IGLV1-56 | CAGTCTGTGCTGACTCAGCCGCCCTCAGTGTCTGGGGC | 14 |
| | CCTGGGCCAGAGGGTCACCATCTCCTGCACTGGAAGCA | |
| | GCTCCAACATCGGGCGTGGTAATTATGTGAACTGGTAC | |
| | CAACAACTCTCAGGAACAGCTCCCAAACTCCTCATCTA | |
| | TGGTAATAGCAATCGACCCTCGGGGGTCCCAGATCGAT | |
| | TTTCTGGCTCCAAGTCTGGCAGCACAGGCTCCTTGACC | |
| | ATCACTGGGCTGCAGGCTGAGGACGAGGCTGATTATTA | |
| | CTGTGCAGCGTGGGACAGCAGTCTCAATGCTCA | |
| IGLV1-42 | CAGTCTGTGCTGACTCAGCCGCCCTCAGTGTCGGGGTC | 15 |
| | CCTGGGCCAGAGGGTCACCATCTCCTGCACTGGAAGCA | |
| | GCTCCAACATCGGGGGTGGTAATTATGTGAGCTGGTAC | |
| | CAACAAGTCCCAGGAACGGCCCCCAGGCTCCTGATTTA | |
| | TGAGAATAACAAACGACCCTCCGGGGTCCCCGATCGA | |
| | TTCTCTGGCTCCAAGTCTGGCAGCTCAGGCTCCCTGAC | |
| | CATCACTGGACTGCAGGCTGACGACGAGGCTGATTATT | |
| | ATTGTGCATCATGGGACGATAGTCTCAGTGCTCA | |
| IGLV1-41 | CAGTCGGGGCCGAATCAACCATCCTCAGTGTCTGGGGC | 16 |
| | CCTGGGCCAGAGGGTCACTATCTCCTGCACTGGAGTTG | |
| | GTAGTTACGTGGGCTGGTACCAACAAATCCCAGGAAT | |
| | GGCCCCCAAAACCATCATCTATGATAATAGCAACCGAC | |
| | CCTCAGGGGTCCCTGATCGATTCTCCGGCTCCAAGTCT | |
| | GGCAGCACAGGCACCCTGACCATCACTGGGCTCCAGG | |
| | CCGAGGACGAGGCTGATTATTACTGCTCATCGTGGGAC | |
| | AGCAGTGGCAGTGCTTA | |
| IGLV3-7 | TCCTATGTGCTGACTCAGCCCCCCTCAGTGTCGGTGAG | 17 |
| | CCTGGGACAGACGGCCAGGACCACCTGTGGAGGAAAC | |
| | AACATTGAAAGCAAAAGTGTTCACTGGTACCAGCAGA | |
| | AGTCAGGCCAAACCCCTGTGCTGATTATCTATAGTGAT | |
| | AGCAACCGGCCCTCAGGAATCCCCGACCGATTCTCAGG | |
| | CACCAACTCGGGGAACACGGCCACCCTGACCATCAGC | |
| | GGGGCCCGGGCCGAGGACGAGGCTGACTATTACTGTC | |
| | AGGTGTGGGACAGTAGTAGTGATGCT | |
| IGLV3-5 | TCAAACAGGGTGACTCAGCCCCCTTCCATTTCAGTGGC | 18 |
| | CCTGGGAGAGATGGCAAGGATCACCTGTGAGGGAAAC | |
| | AACATCGGAAATACATATGTTTCCTGGTACCAGCAGAA | |
| | GCCGAACCAGGTGCCCCTGATGATTATTTATCAGGATA | |
| | GCAACCGGCCTTCAGGGATCCCTGACCGATTCTCTGGC | |
| | TCTAACTCTGGGAACACGGCCACCCTGACTGTCAGCGG | |
| | GGCCCGGGCTGAGGATGAGGCTGACTATTACTGTCTGT | |
| | CTGCTCACAGCAGTAGTAACGTT | |
| IGLV3-13 | TCCTATGTGCTGACTCAGCCCCCATCAGTGGCAGTGAA | 19 |
| | CCTGGGACAGACGGCCAGGATCACATGTGGAGGAAAC | |
| | AACATTGGAAGTAGTTATGCTTACTGGTACCAGCAGAA | |
| | GTCAGGCCAGGCCCCTGTGCTGATTATCTATAAGGATA | |
| | GCAACCGGCCCTCAGGGATCCCTGACCGATTCTCAGGC | |
| | ACCAACTCGGGGAACACGGCCACCCTGACCATCAGCG | |
| | GGGCCCGGGCCGAGGACGAGGCTGACTATTACTGTCA | |
| | GTCATATGACAGCAACTATGATCCT | |
| IGLV1-126 | CAGTCAGGACTGAATCAACCATCCTCAGTGTCTGGGGC | 20 |
| | CCTGGGCCAGAGGGTCACTATCTCCTGCACTGGAATTG | |
| | GTAGTTACGTGGGCTGGTACCAACAAATCCCAGGAAT | |
| | GGTCCCCAAAACCATCATCTATGATAATAGCAACCGAC | |
| | CCTCAGGGGTCCCTGATCGATTCTCCGGCTCCAAGTCT | |
| | GGCAGCACAGGCACTCTGACCATCACTGGGCTCCAGG | |
| | CCGAAGACGAGGCTGATTATTACTGCTCATCGTGGGGC | |
| | GGTGACATTATTGA | |
| IGLV1-N1 | ATGGCCCGGTTCCCTCTTCTCCTGACCCTTCTCATCCAC | 46 |
| | TGCACAGGTTCCTGGGCCCAGTCTGTGCTGACTCAGCC | |
| | GCCCTCAGTGTCTGGGGCCCTGGGCCAGACGGTCACCA | |
| | TCTCCTGCGCTGGAAGTGCCAACAACATTGGTATTGTT | |
| | GGTGTGAACTGGTACCAGCAGCTTCCAGGAAAGGCCC | |
| | CTAAACTCCTCATCTATGCTAATAACAGACGACCCTCA | |
| | AGTGTTCCTGACAGATTTTCTGGCTCCAAGTCTGGCAA | |
| | CACAGGCTCCTTGACCATCACTGGGCTCCAGGCCGAGG | |
| | ACGAGGCTGATTATTACTGCATGTCTGCTGACATTACT | |
| | CAAGGTGCTGA | |
| IGLV1-N14 | ATGGCCTGGTCCCCTCTCCTCCTCACTCTCCTCGCTCAC | 47 |
| | TGCACAGGGATTTGGGCTCAGTCAGGACTGAATCAACC | |
| | ATCCTCAGTGTCTGGGGCCCTGGGCCAGAGGGTCACTA | |
| | TCTCCTGCACTGGAGCTGGTAGTTACGTGGGCTGGTAC | |
| | CAACAAATCCCAGGAATGGCCCCCAAAACCATCATCT | |
| | ATGATAATAGCAACCGACCCTCAGGGGTCCCTGATCGA | |
| | TTCTCCGGCTCCAAGTCTGGCAGCACAGGCACCCTGAC | |
| | CATCACTGGGCTCCAGGCCGAGGACGAGGCTGATTATT | |
| | ACTGCTCATCGTGGGACAGCAGTGGCACAGCTTA | |
| KAPPA V | | |
| IGKV4-1 | GCGATCACGATGACGCAGTCTCCAGGCTCCCTGGCTGG | 21 |
| | GTCTCCAGGTCAGCAGGTCACCATGAACTGCAGGGCC | |
| | AGTCAGAGTGTTAGCAGCTACTTAGCCTGGTACCAGCA | |
| | GAAACCAGGGCAGCATCCTAAGCTGCTCATCTACTCAG | |
| | CTTCCACCCGGGCATCTGGAGTCCCCGACCGATTCAGT | |
| | GGCAGTGGGTCCGGGACGGATTTCACCCTCACCATCAG | |
| | CAACCTCCAGGCTGAAGACGTGGCGAGTTACTACTGTC | |
| | AGCAGTATTACAGCTCTCCTCC | |
| IGKV2-12 | GATATTGTGATGACGCAGACCCCTCTGTCCCTGTCCGT | 22 |
| | CACCCCTGGAGAGCCAGCCTCAATCTCCTGCAGGGCCA | |
| | GTCAGAGCCTCCTGCACAGTGATGGAAATACTTATCTG | |
| | AATTGGTACCTGCAGAAGCCAGGCCAGTCTCCACGGC | |
| | GCTTGATCTATCTTGTTTCCAACCGGGACTCTGGGGTC | |
| | CCAGACAGGTTCAGTGGCAGCGGGTCAGGGACAGATT | |
| | TCACCCTGAGAATCAGCAGGGTGGAGGCTGACGACGT | |
| | CGGTGTTTATTACTGCGGTCAAGCTTTACAGTATCCTCC | |
| IGKV2-5 | GATGTCGTGATGACGCAGACCCCTCTGTCCCTGCCCGT | 23 |
| | CACCCCTGGAGAGCCGGCCTCAATCTCCTGCAGGGCCA | |
| | GTCAGAGCCTCCTGCACAGTAATGGAAATACTTATCTG | |
| | AATTGGTACCTGCAGAAGCCAGGCCAGTCTCCACGGCT | |
| | CCTGATCTATCAGGTTTCCAACCGGGCCTCTGGGGTCC | |
| | CAGACAGGTTCAGTGGCAGCGGGTCAGGGACAGATTT | |
| | CACCCTGAGAATCAGCAGGGTGGAGGCTGACGACGTC | |
| | GGAGTTTATTACTGCCAGCAAGATACACATGCTCCTCG | |
| IGKV2-13 | GATGTCGTGATGACACAGACCCCTCTGTCCCTGCCTGT | 24 |
| | CACCCCTGGAGAGCCGGCCTCAATCTCCTGCAGGGCCA | |
| | GTCAGAGCCTCCTGCACAGTGATGGAAATACTTATCTG | |
| | AATTGGTACCTGCAGAAGCCAGGCCAGTCTCCACGGC | |
| | GACTGATCTATAAGGTTTCCAACCGGGACTCTGGGGTC | |
| | CCAGACAGGTTCAGTGGCAGCGGGTCAGGGACAGATT | |
| | TCACCCTGAGAATCAGCAGGGTAGAGGCTGACGACGT | |
| | CGGAGTTTATTACTGCCTGCAAGGTACAAAGTATCCTA | |
| | C | |
| IGKV2-9 | GATGTCGTGATGACGCAGACCCCTCTGTCCCTGCCCGT | 25 |
| | CACCCCTGGAGAGCCGGCCTCAATCTCCTGCAGGGCCA | |
| | GTCAGAGCCTCCTGCACAGTAATGGAAACACCTATTTA | |
| | CATTGGTACCTGCAGAAGCCAGGCCAGTCTCCACGGCG | |
| | ACTGATCTATAGGGTTTCCAACCGGGACTCTGGGGTCC | |
| | CAGACAGGTTCAGTGGCAGCGGGTCAGGGACAGATTT | |
| | CACCCTGAGAATCAGCAGGGTGGAGGCTGACGACGTC | |
| | GGAGTTTATTACTGCCTGCAAGGTACACACAGACCTCC | |
| IGKV2-4 | GATGTCGTGATGACGCAGACCCCTCTGTCCCTGCCCGT | 26 |
| | CACCCCTGGAGAGCCGGCCTCAATCTCCTGCAGGGCCA | |
| | GTCAGAGCCTCCTGCACAGTAATGGAAATACTTATCTG | |
| | AATTGGTACCTGCAGAAGCCAGGCCAGTCTCCACGGC | |
| | GACTGATCTATAAGGTTTCCAACCGGGACTCTGGGGTC | |
| | CCAGACAGGTTCAGTGGCAGCGGGTCAGGGACAGATT | |
| | TCACCCTGAGAATCAGCAGGGTGGAGGCTGACGACGT | |
| | CGGAGTTTATTACTGCCAGCAAGGTACACATGCTCCTC | |
| | G | |

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, ABAB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention.

The present invention is described in more detail in the following non limiting Examples.

### EXAMPLES

### Example 1: Analysis of Healthy Cat PBMCs, Sorting and 10× Immune Profiling of Intact Single B Cells to Generate VH:VL Pairing Data

2 frozen PBMC samples from 2 healthy European short hair SPF cats were obtained from Life Link Biologicals.

The cellular fraction was enriched for viable lymphocyte cells (including B cells) using FACS sorting of the forward scatter-side scatter lymphocyte gate after gating out dead cells that were stained with a viability dye. Sample 1 generated approximately 320,000 B cells (CD21 positive cells), while sample 2 generated approximately 120,000 B cells (CD21 positive cells). 40,000 cells from each sample were pelleted by centrifugation and processed using the microfluidics encapsulation protocol from the 10× Genomics Chromium Next Gem Single Cell 5′ v2 kit (10× Genomics), following the manufacturer's protocol. The resulting cDNA was quality controlled by quantitation using the Qubit fluorometer (ThermoFisher) and bioanaylser analysis (Agilent). Following this, the cDNA was processed to generate 5′ VDJ NGS sequencing libraries using cat constant-region specific PCR primers as set out in Table 3 designed to cross react with as many constant regions as possible. After pelleting we expect the number of cells loaded into the 10× chip to be approximately 28,000.

The 5′VDJ libraries thus generated were sequenced on an Illumina NGS platform using 150 bp paired-end sequencing. With this read length, a minimum of two thousand read pairs are recommended per cell barcode. The resulting sequencing data was demultiplexed and each library will contain a Read1 and Read2 .fastq.gz pair of files.

The resulting set of FASTQ files was processed via the Cell Ranger software (10× Genomics): for the combination of VDJ and 5′GEX libraries, it was processed via ‘cellranger multi’ command, given the list of heavy and light V+D+J+C reference sequences in the cat repertoire, supplied as reference sets to an internal cat genome reference and annotation (which incorporates information from FelisCatus8.0 and FelisCatus9.0 as well as internal sequence data), including the list of validated IG genes in the 3 IG loci in the cat genome from internal curation, as well as the list of inner primers designed for this purpose as a parameter. The results of the VDJ library through the ‘cellranger multi’ step were QCed and compared with the estimated number of cells in the cell counting step. 14,562 10× V-J paired cells were recovered for sample 1 and 1,503 for sample 2. The reconstructed chains from the Cellranger (all_contig.fasta) were blasted against the set of heavy and light V+D+J reference sequences formatted for running enclone software (10× Genomics). The results of the enclone step were sorted by aa % and filtered for the v_name sequence corresponding to the highly confident heavy+light V repertoire cell barcode calls, with only the highly confident cells chosen as the final result.

### Results:

Heavy chain usage was plotted vs light chain usage from the heavy+light paired data obtained. Some heavy/light chain pairing patterns appear more predominantly than others.

Tables 2a-2c show the combined data in which the top pairings, heavy chain V gene segments and light chain V gene segments are ranked.

Table 4 shows the ranking of light chain V genes by number of different heavy V gene pairings.

TABLE 2a

| Heavy Chain | Light Chain | Frequency |
|---|---|---|
| IGHV3-14 | IGLV1-104 | 510 |
| IGHV3-14 | IGLV1-124 | 428 |
| IGHV3-14 | IGLV1-36 | 418 |
| IGHV3-14 | IGLV1-56 | 333 |
| IGHV3-14 | IGLV1-126 | 319 |
| IGHV3-14 | IGLV1-42 | 312 |
| IGHV3-14 | IGLV1-41 | 300 |
| IGHV3-14 | IGKV4-1 | 271 |
| IGHV3-14 | IGLV3-7 | 266 |
| IGHV3-14 | IGKV2-12 | 237 |
| IGHV3-12 | IGLV1-104 | 236 |
| IGHV3-11 | IGLV1-56 | 232 |
| IGHV3-14 | IGLV1-33 | 224 |
| IGHV3-11 | IGLV1-41 | 222 |

TABLE 2b

| Heavy Chain | Frequency |
|---|---|
| IGHV3-14 | 8712 |
| IGHV3-11 | 5625 |
| IGHV3-12 | 4598 |
| IGHV3-84 | 3923 |
| IGHV3-20 | 2879 |
| IGHV3-27 | 2772 |
| IGHV4-3 | 2746 |
| IGHV3-N3 | 2657 |
| IGHV3-5 | 2424 |

TABLE 2c

| Light Chain | Frequency |
|---|---|
| IGLV1-124 | 2534 |
| IGLV1-36 | 2266 |
| IGLV1-104 | 2239 |
| IGLV1-33 | 2170 |
| IGKV4-1 | 2130 |
| IGLV1-56 | 2071 |
| IGLV1-42 | 2049 |
| IGLV1-41 | 1918 |
| IGLV3-7 | 1918 |
| IGLV3-5 | 1719 |
| IGLV3-13 | 1698 |
| IGKV2-12 | 1670 |
| IGLV1-126 | 1630 |
| IGKV2-5 | 1143 |
| IGKV2-13 | 870 |
| IGKV2-9 | 581 |
| IGKV2-4 | 424 |

TABLE 3

| 5′-3′ primer sequence | Name | SEQ ID NO |
|---|---|---|
| Heavy-IgG | | |
| GTGGGAACACCGATGGGGC | flgG(1-3)_IN-Short | 27 |
| TCACCGGCTCAGGGAAGTAG | flgG(1-3)_IN-Long | 28 |
| ATGCTGCTGAGAGAGTAGAGC | flgG(1-3)_OUT | 29 |
| Heavy-IgA | | |
| TGGGGCTCCAAGTCACTCTC | flgA_IN_Long | 30 |
| GGTTCCCGGAAGCAGACTTC | flgA_OUT | 31 |
| CTGAGGCTCAGCGGGAAGAC | flgA_IN_Short | 32 |
| Light-Lambda | | |
| GGAGGGCTTGGTGGTCTCC | flgL_OUT | 33 |
| TCAAGCCGCTGGGGTAGAAG | flgL_IN_Long | 34 |
| AGGGTGGGAAGAGTGTGACC | flgL_IN_Short | 35 |
| Light-Kappa | | |
| GGACTACGCCATCCACTTTCC | flgK_IN_Long | 36 |
| GGTGCTGCTGAGGCTGTAGG | flgK_OUT | 37 |
| GCACACGATAGAGGCACTTCC | flgK_IN_Short | 38 |
| Heavy-IgM | | |
| TCTCAGGACTGGAGGGAAGG | flgM_OUT | 39 |
| GAGAAGGTGACGGAGCTGGG | flgM_IN-Long | 40 |
| GGACAGGGAGCTCTCACAGG | fIgM_IN-Short | 41 |
| Heavy-IgD | | |
| GGGCAGTCAGGAAGGACAGC | flgD_OUT | 42 |
| CACCTGCACTGACTCTTGGG | flgD_IN-Long | 43 |
| TGACACCCCGAGACCAAGGG | flgD_IN_Short | 44 |

TABLE 4

| Light Chain | Number of different heavy chain pairings |
|---|---|
| IGLV1-124 | 40 |
| IGLV3-7 | 40 |
| IGKV2-5 | 40 |
| IGLV1-42 | 39 |
| IGLV1-104 | 39 |
| IGLV1-56 | 39 |
| IGKV2-9 | 38 |
| IGLV1-126 | 38 |
| IGKV2-12 | 38 |
| IGKV2-13 | 38 |
| IGLV3-13 | 38 |
| IGLV3-5 | 37 |
| IGKV4-1 | 37 |
| IGLV1-36 | 37 |
| IGLV1-41 | 37 |
| IGLV1-33 | 36 |
| IGKV2-4 | 34 |

This experiment was performed again at a later data, and the following data were generated:

TABLE 5a

| Heavy Chain | Usage |
|---|---|
| IGHV3-14 | 14.22% |
| IGHV3-11 | 8.52% |
| IGHV3-12 | 7.77% |
| IGHV3-84 | 5.22% |
| IGHV3-27 | 4.67% |
| IGHV4-3 | 4.60% |
| IGHV3-5 | 4.09% |
| IGHV3-20 | 3.02% |
| IGHV3-16 | 2.77% |

TABLE 5b

| Lambda Chain | Frequency |
|---|---|
| IGLV1-124 | 4.54% |
| IGLV1-36 | 4.20% |
| IGLV1-N1 | 3.87% |
| IGLV1-42 | 3.33% |
| IGLV1-56 | 3.31% |
| IGLV3-7 | 3.21% |
| IGLV1-N14 | 2.96% |
| IGLV3-13 | 2.76% |
| IGLV3-5 | 2.62% |
| IGLV1-104 | 2.23% |
| IGLV1-126 | 0.13% |

TABLE 5c

| Lambda Chain | Frequency |
|---|---|
| IGKV4-1 | 3.12% |
| IGKV2-12 | 2.39% |
| IGKV2-5 | 1.63% |
| IGKV2-13 | 1.24% |
| IGKV2-9 | 0.81% |
| IGKV2-4 | 0.59% |

The invention is further described in the following numbered clauses:
1. A rodent or rodent cell having a genome comprising:
   no more than 9 cat IGHV gene segments, one or more cat IGH D region gene segment(s) and one or more cat IGH J region gene segment(s),
   wherein the rodent or rodent cell is capable of expressing the cat gene segments to form an antibody heavy chain,
   wherein at least one of the cat IGH V gene segments is selected from the list comprising V3-14, V3-11, V3-12, V3-84, V3-20, V3-27, V4-3, V3-N3 and V3-5.
2. A rodent or rodent cell according to claim 1 comprising cat IGH
   (i) V3-14, optionally with up to 8 other cat IGH V gene segments;
   (ii) V3-14 and V3-11, optionally with up to 7 other cat IGH V gene segments:
   (iii) V3-14, V3-11 and V3-12, optionally with up to 6 other cat IGH V gene segments;
   (iv) V3-14, V3-11, V3-12, and V3-84, optionally with up to 5 other cat IGH V gene segments:
   (v) V3-14, V3-11, V3-12, V3-84, and V3-20, optionally with up to 4 other cat IGH V gene segments;
   (vi) V3-14, V3-11, V3-12, V3-84, V3-20, and V3-27, optionally with up to 3 other cat IGH V gene segments:
   (vii) V3-14, V3-11, V3-12, V3-84, V3-20, V3-27, and V4-3, optionally with up to 2 other cat IGH V gene segments;
   (viii) V3-14, V3-11, V3-12, V3-84, V3-20, V3-27, V4-3, and V3-N3, optionally with up to 1 other cat IGH V gene segment: or
   (ix) V3-14, V3-11, V3-12, V3-84, V3-20, V3-27, V4-3, V3-N3 and V3-5.
3. A rodent or rodent cell according to claim 1 comprising no more than 8, no more than 7, no more than 6, no more than 5, no more than 4, no more than 3, no more than two or only 1 of the cat IGH V gene segment(s), optionally wherein the rodent or rodent cell has a genome encoding only a single type of cat heavy chain VDJ variable region.
4. A rodent or rodent cell according to claim 1 comprising at least 2 IGH gene segments, such as at least 3, at least 4, at least 5, at least 6, at least 7, at least 8 or 9 cat IGH V gene segments.
5. A rodent or rodent cell according to claim 1-4 comprising at least one gene segment from cat IGH V gene segment of family 3 and 4.
6. A rodent or rodent cell having a genome comprising:
   no more than 11 cat lambda V gene segments and one or more cat lambda J region gene segment(s);
   wherein the rodent or rodent cell is capable of expressing the cat lambda gene segments to form an antibody light chain,
   wherein at least one of the cat lambda gene segments is selected from V1-124, V1-36, V1-104, V1-33, V1-56, V1-42, V1-41, V3-7, V3-5, V3-13 and V1-126.
7. A rodent or rodent cell according to claim 6 comprising cat lambda V
   (i) V1-124, optionally with up to 10 other cat lambda V gene segments;
   (ii) V1-124 and V1-36, optionally with up to 9 other cat lambda V gene segments;
   (iii) V1-124, V1-36, and V1-104, optionally with up to 8 other cat lambda V gene segments;
   (iv) V1-124, V1-36, V1-104, and V1-33, optionally with up to 7 other cat lambda V gene segments;
   (v) V1-124, V1-36, V1-104, V1-33, and V1-56, optionally with up to 6 other cat lambda V gene segments;
   (vi) V1-124, V1-36, V1-104, V1-33, V1-56, V1-42, optionally with up to 5 other cat lambda V gene segments; or
   (vii) V1-124, V1-36, V1-104, V1-33, V1-56, V1-42, and V1-41, optionally with up to 4 other cat lambda V gene segments.
8. A rodent or rodent cell according to claim 6 comprising no more than 10, no more than 9, no more than 8, no more than 7, no more than 6, no more than 5, no more than 4, no more than 3, no more than 2 or only 1 V gene segment, optionally wherein the rodent or rodent cell has a genome encoding only a single type of cat lambda light chain VJ variable region.
9. A rodent or rodent cell according to claim 6 comprising at least 2 cat lambda V gene segments, such as at least 3, at least 4, at least 5, at least 6, at least 7, at least 8, at least 9, at least 10, or 11 cat lambda V gene segments.
10. A rodent or rodent cell according to claim 6-9 comprising a cat lambda V gene segment from each of the lambda V families 1 and 3.
11. A rodent or rodent cell having a genome comprising:
   no more than 6 cat kappa V gene segments and one or more cat kappa J region gene segment(s),
   wherein the rodent or rodent cell is capable of expressing the cat kappa gene segments to form an antibody light chain,
   wherein at least one of the cat kappa gene segments is selected from V4-1, V2-12, V2-5, V2-13, V2-9 and V2-4.
12. A rodent or rodent cell according to claim 11 comprising cat kappa V
   (i) V4-1, optionally with up to 5 other cat kappa V gene segments;
   (ii) V4-1 and V2-12, optionally with up to 4 other cat kappa V gene segments;
   (iii) V4-1, V2-12, and V2-5, optionally with up to 3 other cat kappa V gene segments;
   (iv) V4-1, V2-12, V2-5, and V2-13, optionally with up to 2 other cat kappa V gene segments;
   (v) V4-1, V2-12, V2-5, V2-13, and V2-9, optionally with up to 1 other cat kappa V gene segment; or
   (vi) V4-1, V2-12, V2-5, V2-13, V2-4 and V2-4; or
   (vii) V4-1, V2-12, V2-5, V2-13, V2-9 and V2-4.
13. A rodent or rodent cell according to claim 11 comprising no more than 5, no more than 4, no more than 3, no more than 2, or only 1 cat kappa V gene segment, optionally wherein the rodent or rodent cell has a genome encoding only a single type of cat kappa light chain VJ variable region.
14. A rodent or rodent cell according to claim 11 comprising at least 2 cat kappa V gene segments, such as at least 3, at least 4, at least 5, or 6 cat kappa V gene segments.
15. A rodent or rodent cell according to claim 11-14 comprising a cat kappa V gene segment from each of the kappa V gene segment families 2 and 4, suitably including IGKV4-1 and IGKV2-12.
16. The rodent or rodent cell of claims 1-15 wherein the rodent genome comprises cat gene segments from both the heavy chain and at least one cat light chain, optionally wherein
   (i) the rodent or cell genome is according to claim 1-5 in combination with cat lambda gene segments according to any one of claims 6-10, and/or cat kappa V gene segments according to any of claims 11-15 or
   (ii) the rodent or cell genome is according to claim 1-5 and additionally comprises cat kappa V and J gene segments capable of being expressed to form a kappa antibody light chain variable domain; and/or the rodent or cell genome is a rodent or rodent cell according to claim 1-4 additionally comprising cat lambda V and J gene segments capable of being expressed to form a lambda antibody light chain.
17. The rodent or rodent cell of any preceding claim wherein
   (i) the cat gene segments(s) are located in the genome upstream of a rodent constant region, suitably upstream of the heavy chain constant region for inserted cat heavy chain variable region gene segments and/or suitably upstream of a light chain constant region for inserted cat light chain variable region gene segments, such that the rodent or rodent cell is able to produce a chimaeric antibody heavy chain, or light chain, or both, resulting from expression of the inserted cat variable region gene segments and a host constant region: or
   (ii) the cat gene segments(s) are located in the genome in functional arrangement with a cat constant region, such that the rodent is able to produce an antibody chain resulting from the expression of the cat VDJ gene segments with the cat constant region, and/or an antibody chain resulting from the expression of the cat VJ gene segments with the cat constant region.
18. A rodent or rodent cell according to any one of claims 1-16 having a genome in which one or more cat V gene segments are regulated by a non-coding regulatory sequence, which is one or more of: a promoter preceding each V gene segment coding sequence, intron, splice site, and recombination signal sequences for V(D)J recombination, wherein the regulatory sequence is of endogenous host origin.
19. A nucleic acid encoding a chimaeric antibody chain, the nucleic acid having a variable domain which comprises any one of the cat IGHV gene segments selected from the list comprising V3-14, V3-11, V3-12, V3-84, V3-20, V3-27, V4-3, V3-N3 and V3-5 and a rodent constant region, optionally wherein the cat V gene segment has undergone somatic hypermutation in a rodent.
20. An antibody chain having a variable region obtained or obtainable by expression of any one of cat IGH V3-14, V3-11, V3-12, V3-84, V3-20, V3-27, V4-3, V3-N3 and V3-5 in a rodent, in conjunction with a rodent constant region.
21. A nucleic acid encoding a chimaeric antibody chain, the nucleic acid having a variable domain which comprises any one of the cat lambda V gene segments selected from the list comprising lambda V1-124, V1-36, V1-104, V1-33, V1-56, V1-42, V1-41, V3-7, V3-5, V3-13 and V1-126, and a rodent constant region, optionally wherein the cat V gene segment has undergone somatic hypermutation in a rodent.
22. An antibody chain having a variable region obtained or obtainable by expression of any one of cat lambda V1-124, V1-36, V1-104, V1-33, V1-56, V1-42, V1-41, V3-7, V3-5, V3-13 and V1-126 in a rodent, in conjunction with a rodent constant region.
23. A nucleic acid encoding a chimaeric antibody chain, the nucleic acid having a variable domain which comprises any one of the cat kappa V gene segments selected from the list comprising V4-1, V2-12, V2-5, V2-13, V2-9 and V2-4 and a rodent constant region, optionally wherein the cat V gene segment has undergone somatic hypermutation in a rodent.
24. An antibody chain having a variable region obtained or obtainable by expression of any one of cat kappa V4-1, V2-12, V2-5, V2-13, V2-9 and V2-4 in a rodent, in conjunction with a rodent constant region.
25. A pharmaceutical composition comprising an antibody chain having a variable region comprising any one of cat IGH V3-14, V3-11, V3-12, V3-84, V3-20, V3-27, V4-3, V3-N3 and V3-5, in conjunction with a pharmaceutically acceptable excipient or carrier.
26. A pharmaceutical composition comprising an antibody chain having a variable region comprising any one of cat lambda V1-124, V1-36, V1-104, V1-33, V1-56, V1-42, V1-41, V3-7, V3-5, V3-13 and V1-126 in conjunction with a pharmaceutically acceptable excipient or carrier.
27. A pharmaceutical composition comprising an antibody chain having a variable region comprising any one of cat kappa V4-1, V2-12, V2-5, V2-13, V2-9 and V2-4, in conjunction with a pharmaceutically acceptable excipient or carrier.
28. A pharmaceutical composition comprising an antibody, the antibody having
   (i) a heavy chain having a variable region obtained by expression of any one of cat IGH V3-14, V3-11, V3-12, V3-84, V3-20, V3-27, V4-3, V3-N3 and V3-5, and either
   (ii) a lambda light chain obtained by expression of any one of cat lambda V1-124, V1-36, V1-104, V1-33, V1-56, V1-42, V1-41, V3-7, V3-5, V3-13 and V1-126; or
   (iii) a kappa light chain obtained by expression of any one of cat kappa V4-1, V2-12, V2-5, V2-13, V2-9 and V2-4;
   the antibody being formulated in conjunction with a pharmaceutically acceptable excipient or carrier.
29. An antibody heavy chain repertoire, the repertoire comprising antibody heavy chains having cat IGH V gene segments from no more than 9 different cat IGH V gene segments, wherein at least one of the cat IGH V gene segments is selected from the list comprising V3-14, V3-11, V3-12, V3-84, V3-20, V3-27, V4-3, V3-N3 and V3-5
   optionally wherein the repertoire comprises
   A antibody heavy chains having cat IGH V gene segments expressed from
   (i) V3-14, optionally with up to 8 other cat IGH V gene segments;
   (ii) V3-14 and V3-11, optionally with up to 7 other cat IGH V gene segments;
   (iii) V3-14, V3-11 and V3-12, optionally with up to 6 other cat IGH V gene segments;
   (iv) V3-14, V3-11, V3-12, and V3-84, optionally with up to 5 other cat IGH V gene segments;
   (v) V3-14, V3-11, V3-12, V3-84, and V3-20, optionally with up to 4 other cat IGH V gene segments;
   (vi) V3-14, V3-11, V3-12, V3-84, V3-20, and V3-27, optionally with up to 3 other cat IGH V gene segments;
   (vii) V3-14, V3-11, V3-12, V3-84, V3-20, V3-27, and V4-3, optionally with up to 2 other cat IGH V gene segments;
   (viii) V3-14, V3-11, V3-12, V3-84, V3-20, V3-27, V4-3, and V3-N3, optionally with up to 1 other cat IGH V gene segment; or
   (ix) V3-14, V3-11, V3-12, V3-84, V3-20, V3-27, V4-3, V3-N3 and V3-5;
   and/or
   B antibody heavy chains expressed from IGH V gene segments of cat IGH V families 3 and 4.
30. An antibody light chain repertoire, the repertoire comprising antibody light chains having cat lambda V gene segments from no more than 11 different cat lambda V gene segments, wherein at least one of the cat lambda V gene segments is selected from the list comprising V1-124, V1-36, V1-104, V1-33, V1-56, V1-42, V1-41, V3-7, V3-5, V3-13 and V1-126,
   optionally wherein the repertoire comprises
   A antibody lambda light chains having cat lambda V gene segments including
   (i) V1-124, optionally with up to 10 other cat lambda V gene segments;
   (ii) V1-124 and V1-36, optionally with up to 9 other cat lambda V gene segments;
   (iii) V1-124, V1-36, and V1-104, optionally with up to 8 other cat lambda V gene segments;
   (iv) V1-124, V1-36, V1-104, and V1-33, optionally with up to 7 other cat lambda V gene segments;
   (v) V1-124, V1-36, V1-104, V1-33, and V1-56, optionally with up to 6 other cat lambda V gene segments;
   (vi) V1-124, V1-36, V1-104, V1-33, V1-56, V1-42, optionally with up to 5 other cat lambda V gene segments; or
   (vii) V1-124, V1-36, V1-104, V1-33, V1-56, V1-42, and V1-41, optionally with up to 4 other cat lambda V gene segments;
   and/or
   B antibody lambda light chains expressed from cat lambda V gene segments of cat lambda families 1 and 3.
31. An antibody light chain repertoire, the repertoire comprising antibody kappa light chains having cat kappa V gene segments from no more than 5 different cat kappa V gene segments, wherein at least one of the cat kappa V gene segments is selected from the list comprising V4-1, V2-12, V2-5, V2-13, V2-9 and V2-4,
   optionally wherein the repertoire comprises
   A antibody kappa light chains having cat kappa V gene segments including
   (i) V4-1, optionally with up to 5 other cat kappa V gene segments;
   (ii) V4-1 and V2-12, optionally with up to 4 other cat kappa V gene segments;
   (iii) V4-1, V2-12, and V2-5, optionally with up to 3 other cat kappa V gene segments;
   (iv) V4-1, V2-12, V2-5, and V2-13, optionally with up to 2 other cat kappa V gene segments;
   (v) V4-1, V2-12, V2-5, V2-13, and V2-9, optionally with up to 1 other cat kappa V gene segment; or
   (vi) V4-1, V2-12, V2-5, V2-13, V2-4 and V2-4; or
   (vii) V4-1, V2-12, V2-5, V2-13, V2-9 and V2-4;
   and/or
   B antibody kappa light chains expressed from cat kappa V gene segments of cat kappa V families 2 and 4.
32. An antibody or antibody chain, or part thereof, or a DNA encoding an antibody chain or a part thereof, which has been obtained or is obtainable from a rodent or rodent cell according to any one of claims 1-18 or from a repertoire according to any one of claims 29-31.
33. An antibody or antibody or chain, or part thereof, obtained or obtainable from a rodent or cell according to any one of claims 1-18, or from a repertoire according to any one of claims 29-31, for use in treatment or prevention of disease a cat in need thereof.
34. A method for producing a rodent or rodent cell as claimed in any of the preceding claims, the method comprising inserting into a rodent cell genome
   (i) no more than 9 cat IGH V gene segments, one or more cat IGH D region gene segments and one or more cat IGH J region gene segments, wherein at least one of the cat IGH V gene segments is selected from the list comprising V3-14, V3-11, V3-12, V3-84, V3-20, V3-27, V4-3, V3-N3 and V3-5; and/or
   (ii) no more than 11 cat lambda V gene segments and one or more cat lambda J region gene segments, wherein at least one of the cat V gene segments is selected from the list comprising V1-124, V1-36, V1-104, V1-33, V1-56, V1-42, V1-41, V3-7, V3-5, V3-13 and V1-126; and/or
   (iii) no more than 6 cat kappa V gene segments and one or more cat kappa J region gene segments, wherein at least one of the cat kappa gene segments is selected from V4-1, V2-12, V2-5, V2-13, V2-9 and V2-4;
   and wherein the rodent or rodent cell is capable of expressing the cat variable region gene segment or segments in combination with a constant region to form an antibody chain.
35. A method for producing an antibody or antibody chain specific to a desired antigen, the method comprising immunizing a rodent according to any of claims 1-18 with the desired antigen and recovering the antibody chain or antibody or recovering a cell producing the antibody chain or antibody.
36. A method for producing an antibody chain or antibody specific to a desired antigen the method comprising immunizing a rodent according to any of claims 1-18 with the desired antibody and then replacing the rodent constant region of the antibody chain or antibody with a cat constant region, suitably by engineering of the nucleic acid encoding the antibody.
37. A method for producing an antibody chain or a part thereof, the antibody chain having a cat variable region, the method comprising expressing a nucleic acid encoding the antibody chain, or part thereof in a cell,
   wherein the sequence of the nucleic acid encoding the variable region of the antibody chain is obtained from or obtainable from rodent according to any one of claims 1-18 immunised with an antigen, or obtained from an antibody of an antibody repertoire of claims 29-31, comprising one of the cat heavy, kappa or lambda gene segments according to claim 1, 5 or 10, respectively,
   optionally including the subsequent steps of:
      purifying and/or isolating the antibody chain, and
      optionally then formulating the antibody into a pharmaceutically acceptable formulation suitable for administration into a cat.
38. A method of making a pharmaceutical composition, the method comprising producing an antibody according to the method of any one of claims 35-37, and further comprising combining the antibody with a pharmaceutically acceptable carrier or other excipient to produce the
39. A pharmaceutical composition comprising an antibody according to claims 25-28 which is a monoclonal antibody.
40. A method of treatment of a cat, the method comprising delivery of an antibody or antibody chain or functional part thereof to a cat in need thereof, the antibody being obtained or obtainable from a rodent or cell according to any one of claims 1-18, optionally wherein
   the antibody chain is according to any one of claim 20, 22, 24 or 32, and/or
   the antibody or antibody chain is in the form of a pharmaceutical composition according to any one of claims 25-28, and/or
   the antibody or antibody chain is a fully cat antibody or antibody chain, having a cat variable and constant region;
   preferably wherein the method comprises delivery of an antibody in the form of a pharmaceutical composition having a fully cat heavy chain and a fully cat light chain.
41. A rodent or rodent cell having a genome comprising a cat IGH V gene segment selected from the list comprising IGH V3-14 (SEQ ID NO: 1), V1-36 (SEQ ID NO: 11), V1-104 (SEQ ID NO: 12), and V1-42 (SEQ ID NO: 15), one or more cat IGH D region gene segment(s) and one or more cat IGH J region gene segment(s),
   wherein the rodent or rodent cell is capable of expressing the cat gene segments to form an antibody heavy chain.
42. A rodent or rodent cell having a genome comprising a cat IGK V gene segment IGK V2-5 (SEQ ID NO: 23), and one or more cat IGH J region gene segment(s),
   wherein the rodent or rodent cell is capable of expressing the cat gene segments to form an antibody light chain.

1. A rodent or rodent cell having a genome comprising;
no more than 9 cat IGHV gene segments, one or more cat IGH D region gene segment(s) and one or more cat IGH J region gene segment(s),
wherein the rodent or rodent cell is capable of expressing the cat gene segments to form an antibody heavy chain,
wherein at least one of the cat IGH V gene segments is selected from the list comprising V3-14, V3-11, V3-12, V3-84, V3-20, V3-27, V4-3, V3-16 and V3-5.

2. A rodent or rodent cell according to claim 1 comprising cat IGH
(i) V3-14, optionally with up to 8 other cat IGH V gene segments;
(ii) V3-14 and V3-11, optionally with up to 7 other cat IGH V gene segments;
(iii) V3-14, V3-11 and V3-12, optionally with up to 6 other cat IGH V gene segments;
(iv) V3-14, V3-11, V3-12, and V3-84, optionally with up to 5 other cat IGH V gene segments;
(v) V3-14, V3-11, V3-12, V3-84, and V3-20, optionally with up to 4 other cat IGH V gene segments;
(vi) V3-14, V3-11, V3-12, V3-84, V3-20, and V3-27, optionally with up to 3 other cat IGH V gene segments;
(vii) V3-14, V3-11, V3-12, V3-84, V3-20, V3-27, and V4-3, optionally with up to 2 other cat IGH V gene segments;
(viii) V3-14, V3-11, V3-12, V3-84, V3-20, V3-27, V4-3, and V3-16, optionally with up to 1 other cat IGH V gene segment; or
(ix) V3-14, V3-11, V3-12, V3-84, V3-20, V3-27, V4-3, V3-16 and V3-5.

3. A rodent or rodent cell according to claim 1 comprising no more than 8, no more than 7, no more than 6, no more than 5, no more than 4, no more than 3, no more than two or only 1 of the cat IGH V gene segment(s), optionally wherein the rodent or rodent cell has a genome encoding only a single type of cat heavy chain VDJ variable region.

4. A rodent or rodent cell according to claim 1 comprising at least 2 IGH gene segments, such as at least 3, at least 4, at least 5, at least 6, at least 7, at least 8 or 9 cat IGH V gene segments.

5. A rodent or rodent cell according to claim 1-4 comprising at least one gene segment from cat IGH V gene segment of family 3 and 4.

6. A rodent or rodent cell having a genome comprising:
no more than 11 cat lambda V gene segments and one or more cat lambda J region gene segment(s);
wherein the rodent or rodent cell is capable of expressing the cat lambda gene segments to form an antibody light chain,
wherein at least one of the cat lambda gene segments is selected from V1-124, V1-36, V1-104, V1-N1, V1-56, V1-42, V1-N14, V3-7, V3-5, V3-13 and V1-126.

7. A rodent or rodent cell according to claim 6 comprising cat lambda V
(i) V1-124, optionally with up to 10 other cat lambda V gene segments;
(ii) V1-124 and V1-36, optionally with up to 9 other cat lambda V gene segments;
(iii) V1-124, V1-36, and V1-104, optionally with up to 8 other cat lambda V gene segments;
(iv) V1-124, V1-36, V1-104, and V1-N1, optionally with up to 7 other cat lambda V gene segments;
(v) V1-124, V1-36, V1-104, V1-N1, and V1-56, optionally with up to 6 other cat lambda V gene segments;
(vi) V1-124, V1-36, V1-104, V1-N1, V1-56, V1-42, optionally with up to 5 other cat lambda V gene segments; or
(vii) V1-124, V1-36, V1-104, V1-N1, V1-56, V1-42, and V1-N14, optionally with up to 4 other cat lambda V gene segments.

8. A rodent or rodent cell according to claim 6 comprising no more than 10, no more than 9, no more than 8, no more than 7, no more than 6, no more than 5, no more than 4, no more than 3, no more than 2 or only 1 V gene segment, optionally wherein the rodent or rodent cell has a genome encoding only a single type of cat lambda light chain VJ variable region.

9. A rodent or rodent cell according to claim 6 comprising at least 2 cat lambda V gene segments, such as at least 3, at least 4, at least 5, at least 6, at least 7, at least 8, at least 9, at least 10, or 11 cat lambda V gene segments.

10. A rodent or rodent cell according to claim 6-9 comprising a cat lambda V gene segment from each of the lambda V families 1 and 3.

11. A rodent or rodent cell having a genome comprising:
no more than 6 cat kappa V gene segments and one or more cat kappa J region gene segment(s),
wherein the rodent or rodent cell is capable of expressing the cat kappa gene segments to form an antibody light chain,
wherein at least one of the cat kappa gene segments is selected from V4-1, V2-12, V2-5, V2-13, V2-9 and V2-4.

12. A rodent or rodent cell according to claim 11 comprising cat kappa V
(i) V4-1, optionally with up to 5 other cat kappa V gene segments;
(ii) V4-1 and V2-12, optionally with up to 4 other cat kappa V gene segments;
(iii) V4-1, V2-12, and V2-5, optionally with up to 3 other cat kappa V gene segments;
(iv) V4-1, V2-12, V2-5, and V2-13, optionally with up to 2 other cat kappa V gene segments;
(v) V4-1, V2-12, V2-5, V2-13, and V2-9, optionally with up to 1 other cat kappa V gene segment; or
(vi) V4-1, V2-12, V2-5, V2-13, V2-9 and V2-4.

13. A rodent or rodent cell according to claim 11 comprising no more than 5, no more than 4, no more than 3, no more than 2, or only 1 cat kappa V gene segment, optionally wherein the rodent or rodent cell has a genome encoding only a single type of cat kappa light chain VJ variable region.

14. A rodent or rodent cell according to claim 11 comprising at least 2 cat kappa V gene segments, such as at least 3, at least 4, at least 5, or 6 cat kappa V gene segments.

15. A rodent or rodent cell according to claim 11-14 comprising a cat kappa V gene segment from each of the kappa V gene segment families 2 and 4, suitably including IGKV4-1 and IGKV2-12.

16. The rodent or rodent cell of claims 1-15 wherein the rodent genome comprises cat gene segments from both the heavy chain and at least one cat light chain, optionally wherein
(i) the rodent or cell genome is according to claim 1-5 in combination with cat lambda gene segments according to any one of claims 6-10, and/or cat kappa V gene segments according to any of claims 11-15 or
(ii) the rodent or cell genome is according to claim 1-5 and additionally comprises cat kappa V and J gene segments capable of being expressed to form a kappa antibody light chain variable domain; and/or the rodent or cell genome is a rodent or rodent cell according to claim 1-4 additionally comprising cat lambda V and J gene segments capable of being expressed to form a lambda antibody light chain.

17. The rodent or rodent cell of any preceding claim wherein
(i) the cat gene segments(s) are located in the genome upstream of a rodent constant region, suitably upstream of the heavy chain constant region for inserted cat heavy chain variable region gene segments and/or suitably upstream of a light chain constant region for inserted cat light chain variable region gene segments, such that the rodent or rodent cell is able to produce a chimaeric antibody heavy chain, or light chain, or both, resulting from expression of the inserted cat variable region gene segments and a host constant region; or
(ii) the cat gene segments(s) are located in the genome in functional arrangement with a cat constant region, such that the rodent is able to produce an antibody chain resulting from the expression of the cat VDJ gene segments with the cat constant region, and/or an antibody chain resulting from the expression of the cat VJ gene segments with the cat constant region.

18. A rodent or rodent cell according to any one of claims 1-16 having a genome in which one or more cat V gene segments are regulated by a non-coding regulatory sequence, which is one or more of: a promoter preceding each V gene segment coding sequence, intron, splice site, and recombination signal sequences for V(D)J recombination, wherein the regulatory sequence is of endogenous host origin.

19. A nucleic acid encoding a chimaeric antibody chain, the nucleic acid having a variable domain which comprises any one of the cat IGHV gene segments selected from the list comprising V3-14, V3-11, V3-12, V3-84, V3-20, V3-27, V4-3, V3-16 and V3-5 and a rodent constant region, optionally wherein the cat V gene segment has undergone somatic hypermutation in a rodent.

20. An antibody chain having a variable region obtained or obtainable by expression of any one of cat IGH V3-14, V3-11, V3-12, V3-84, V3-20, V3-27, V4-3, V3-16 and V3-5 in a rodent, in conjunction with a rodent constant region.

21. A nucleic acid encoding a chimaeric antibody chain, the nucleic acid having a variable domain which comprises any one of the cat lambda V gene segments selected from the list comprising lambda V1-124, V1-36, V1-104, V1-N1, V1-56, V1-42, V1-N14, V3-7, V3-5, V3-13 and V1-126, and a rodent constant region, optionally wherein the cat V gene segment has undergone somatic hypermutation in a rodent.

22. An antibody chain having a variable region obtained or obtainable by expression of any one of cat lambda V1-124, V1-36, V1-104, V1-N1, V1-56, V1-42, V1-N14, V3-7, V3-5, V3-13 and V1-126 in a rodent, in conjunction with a rodent constant region.

23. A nucleic acid encoding a chimaeric antibody chain, the nucleic acid having a variable domain which comprises any one of the cat kappa V gene segments selected from the list comprising V4-1, V2-12, V2-5, V2-13, V2-9 and V2-4 and a rodent constant region, optionally wherein the cat V gene segment has undergone somatic hypermutation in a rodent.

24. An antibody chain having a variable region obtained or obtainable by expression of any one of cat kappa V4-1, V2-12, V2-5, V2-13, V2-9 and V2-4 in a rodent, in conjunction with a rodent constant region.

25. A pharmaceutical composition comprising an antibody chain having a variable region comprising any one of cat IGH V3-14, V3-11, V3-12, V3-84, V3-20, V3-27, V4-3, V3-16 and V3-5, in conjunction with a pharmaceutically acceptable excipient or carrier.

26. A pharmaceutical composition comprising an antibody chain having a variable region comprising any one of cat lambda V1-124, V1-36, V1-104, V1-N1, V1-56, V1-42, V1-N14, V3-7, V3-5, V3-13 and V1-126 in conjunction with a pharmaceutically acceptable excipient or carrier.

27. A pharmaceutical composition comprising an antibody chain having a variable region comprising any one of cat kappa V4-1, V2-12, V2-5, V2-13, V2-9 and V2-4, in conjunction with a pharmaceutically acceptable excipient or carrier.

28. A pharmaceutical composition comprising an antibody, the antibody having
(i) a heavy chain having a variable region obtained by expression of any one of cat IGH V3-14, V3-11, V3-12, V3-84, V3-20, V3-27, V4-3, V3-16 and V3-5, and either
(ii) a lambda light chain obtained by expression of any one of cat lambda V1-124, V1-36, V1-104, V1-N1, V1-56, V1-42, V1-N14, V3-7, V3-5, V3-13 and V1-126; or
(iii) a kappa light chain obtained by expression of any one of cat kappa V4-1, V2-12, V2-5, V2-13, V2-9 and V2-4;
the antibody being formulated in conjunction with a pharmaceutically acceptable excipient or carrier.

29. An antibody heavy chain repertoire, the repertoire comprising antibody heavy chains having cat IGH V gene segments from no more than 9 different cat IGH V gene segments, wherein at least one of the cat IGH V gene segments is selected from the list comprising V3-14, V3-11, V3-12, V3-84, V3-20, V3-27, V4-3, V3-16 and V3-5
optionally wherein the repertoire comprises
A antibody heavy chains having cat IGH V gene segments expressed from
(i) V3-14, optionally with up to 8 other cat IGH V gene segments;
(ii) V3-14 and V3-11, optionally with up to 7 other cat IGH V gene segments;
(iii) V3-14, V3-11 and V3-12, optionally with up to 6 other cat IGH V gene segments;
(iv) V3-14, V3-11, V3-12, and V3-84, optionally with up to 5 other cat IGH V gene segments;
(v) V3-14, V3-11, V3-12, V3-84, and V3-20, optionally with up to 4 other cat IGH V gene segments;
(vi) V3-14, V3-11, V3-12, V3-84, V3-20, and V3-27, optionally with up to 3 other cat IGH V gene segments;
(vii) V3-14, V3-11, V3-12, V3-84, V3-20, V3-27, and V4-3, optionally with up to 2 other cat IGH V gene segments;
(viii) V3-14, V3-11, V3-12, V3-84, V3-20, V3-27, V4-3, and V3-16, optionally with up to 1 other cat IGH V gene segment; or
(ix) V3-14, V3-11, V3-12, V3-84, V3-20, V3-27, V4-3, V3-16 and V3-5;
and/or
B antibody heavy chains expressed from IGH V gene segments of cat IGH V families 3 and 4.

30. An antibody light chain repertoire, the repertoire comprising antibody light chains having cat lambda V gene segments from no more than 11 different cat lambda V gene segments, wherein at least one of the cat lambda V gene segments is selected from the list comprising V1-124, V1-36, V1-104, V1-N1, V1-56, V1-42, V1-N14, V3-7, V3-5, V3-13 and V1-126,
optionally wherein the repertoire comprises
A antibody lambda light chains having cat lambda V gene segments including
(i) V1-124, optionally with up to 10 other cat lambda V gene segments;
(ii) V1-124 and V1-36, optionally with up to 9 other cat lambda V gene segments;
(iii) V1-124, V1-36, and V1-104, optionally with up to 8 other cat lambda V gene segments;
(iv) V1-124, V1-36, V1-104, and V1-N1, optionally with up to 7 other cat lambda V gene segments;
(v) V1-124, V1-36, V1-104, V1-N1, and V1-56, optionally with up to 6 other cat lambda V gene segments;
(vi) V1-124, V1-36, V1-104, V1-N1, V1-56, V1-42, optionally with up to 5 other cat lambda V gene segments; or
(vii) V1-124, V1-36, V1-104, V1-N1, V1-56, V1-42, and V1-N14, optionally with up to 4 other cat lambda V gene segments;
and/or
B antibody lambda light chains expressed from cat lambda V gene segments of cat lambda families 1 and 3.

31. An antibody light chain repertoire, the repertoire comprising antibody kappa light chains having cat kappa V gene segments from no more than 5 different cat kappa V gene segments, wherein at least one of the cat kappa V gene segments is selected from the list comprising V4-1, V2-12, V2-5, V2-13, V2-9 and V2-4,
optionally wherein the repertoire comprises
A antibody kappa light chains having cat kappa V gene segments including
(i) V4-1, optionally with up to 5 other cat kappa V gene segments;
(ii) V4-1 and V2-12, optionally with up to 4 other cat kappa V gene segments;
(iii) V4-1, V2-12, and V2-5, optionally with up to 3 other cat kappa V gene segments;
(iv) V4-1, V2-12, V2-5, and V2-13, optionally with up to 2 other cat kappa V gene segments;
(v) V4-1, V2-12, V2-5, V2-13, and V2-9, optionally with up to 1 other cat kappa V gene segment; or
(vi) V4-1, V2-12, V2-5, V2-13, V2-9 and V2-4;
and/or
B antibody kappa light chains expressed from cat kappa V gene segments of cat kappa V families 2 and 4.

32. An antibody or antibody chain, or part thereof, or a DNA encoding an antibody chain or a part thereof, which has been obtained or is obtainable from a rodent or rodent cell according to any one of claims 1-18 or from a repertoire according to any one of claims 29-31.

33. An antibody or antibody or chain, or part thereof, obtained or obtainable from a rodent or cell according to any one of claims 1-18, or from a repertoire according to any one of claims 29-31, for use in treatment or prevention of disease a cat in need thereof.

34. A method for producing a rodent or rodent cell as claimed in any of the preceding claims, the method comprising inserting into a rodent cell genome
(i) no more than 9 cat IGH V gene segments, one or more cat IGH D region gene segments and one or more cat IGH J region gene segments, wherein at least one of the cat IGH V gene segments is selected from the list comprising V3-14, V3-11, V3-12, V3-84, V3-20, V3-27, V4-3, V3-16 and V3-5; and/or
(ii) no more than 11 cat lambda V gene segments and one or more cat lambda J region gene segments, wherein at least one of the cat V gene segments is selected from the list comprising V1-124, V1-36, V1-104, V1-N1, V1-56, V1-42, V1-N14, V3-7, V3-5, V3-13 and V1-126; and/or
(iii) no more than 6 cat kappa V gene segments and one or more cat kappa J region gene segments, wherein at least one of the cat kappa gene segments is selected from V4-1, V2-12, V2-5, V2-13, V2-9 and V2-4;
and wherein the rodent or rodent cell is capable of expressing the cat variable region gene segment or segments in combination with a constant region to form an antibody chain.

35. A method for producing an antibody or antibody chain specific to a desired antigen, the method comprising immunizing a rodent according to any of claims 1-18 with the desired antigen and recovering the antibody chain or antibody or recovering a cell producing the antibody chain or antibody.

36. A method for producing an antibody chain or antibody specific to a desired antigen the method comprising immunizing a rodent according to any of claims 1-18 with the desired antibody and then replacing the rodent constant region of the antibody chain or antibody with a cat constant region, suitably by engineering of the nucleic acid encoding the antibody.

37. A method for producing an antibody chain or a part thereof, the antibody chain having a cat variable region, the method comprising expressing a nucleic acid encoding the antibody chain, or part thereof in a cell,
wherein the sequence of the nucleic acid encoding the variable region of the antibody chain is obtained from or obtainable from rodent according to any one of claims 1-18 immunised with an antigen, or obtained from an antibody of an antibody repertoire of claims 29-31, comprising one of the cat heavy, kappa or lambda gene segments according to claim 1, 5 or 10, respectively,
optionally including the subsequent steps of:
purifying and/or isolating the antibody chain, and
optionally then formulating the antibody into a pharmaceutically acceptable formulation suitable for administration into a cat.

38. A method of making a pharmaceutical composition, the method comprising producing an antibody according to the method of any one of claims 35-37, and further comprising combining the antibody with a pharmaceutically acceptable carrier or other excipient to produce the pharmaceutical composition.

39. A pharmaceutical composition comprising an antibody according to claims 25-28 which is a monoclonal antibody.

40. A method of treatment of a cat, the method comprising delivery of an antibody or antibody chain or functional part thereof to a cat in need thereof, the antibody being obtained or obtainable from a rodent or cell according to any one of claims 1-18, optionally wherein
the antibody chain is according to any one of claim 20, 22, 24 or 32, and/or
the antibody or antibody chain is in the form of a pharmaceutical composition according to any one of claims 25-28, and/or
the antibody or antibody chain is a fully cat antibody or antibody chain, having a cat variable and constant region;
preferably wherein the method comprises delivery of an antibody in the form of a pharmaceutical composition having a fully cat heavy chain and a fully cat light chain.

41. A rodent or rodent cell having a genome comprising a cat IGH V gene segment selected from the list comprising IGH V3-14 (SEQ ID NO: 1), V1-36 (SEQ ID NO: 11), V1-104 (SEQ ID NO: 12), and V1-42 (SEQ ID NO: 15), one or more cat IGH D region gene segment(s) and one or more cat IGH J region gene segment(s),
wherein the rodent or rodent cell is capable of expressing the cat gene segments to form an antibody heavy chain.

42. A rodent or rodent cell having a genome comprising a cat IGK V gene segment IGK V2-5 (SEQ ID NO: 23), and one or more cat IGH J region gene segment(s),
wherein the rodent or rodent cell is capable of expressing the cat gene segments to form an antibody light chain.

43. A vector or cell genome or cell line genome or rodent genome comprising cat D gene segment IGHD-N1 (SEQ ID NO: 48).

44. A rodent or rodent cell having a genome comprising:
one or more cat IGHV gene segments, cat D gene segment IGHD-N1, and one or more cat IGH J region gene segment(s),
wherein the rodent or rodent cell is capable of expressing the cat gene segments to form an antibody heavy chain,
optionally wherein there are no more than 9 cat IGHV gene segments and wherein at least one of the cat IGH V gene segments is selected from the list comprising V3-14, V3-11, V3-12, V3-84, V3-20, V3-27, V4-3, V3-16N3 and V3-5.


SEQUENCE LISTING (48 sequences)

SEQ ID NO: 1 - DNA, 296 bp, Felis catus
  source 1..296: mol_type = genomic DNA
gatgtgcagctggtggaatctgggggagacctggtgaagcctggggggtccctgagactcacctgtgtggcctctggattcaccttcagtagctatagcatgaactgggtccgccaggctccagggaaggggctgcagtgggtcgcatatattagtagtagtggaggtagcatatactacgcagactccgtgaagggccgattcaccatctccagagacaacgccaagaacacgctgtatctgcagatgaacagcctgaagaccgaggacacggccacatattactgtgcgagtga

SEQ ID NO: 2 - DNA, 296 bp, Felis catus
  source 1..296: mol_type = genomic DNA
gacgtgcaattggtggagtctgggggagacctggtgaagcctggggggtccctgagactcacctgtgtggcctctggattcaccttcagtagctatgcaatgagctgggtccgccaggctccagggaaggggctgcagtgggtcgcagatattagtagtagtggaggtagcacatactacgcagactccgtgaagggccgattcaccatctccagagacaatgccaagaacacgctgtatctgcagatgaacagcctcaagaccgaggacacggccacatattactgtgcgaaaga

SEQ ID NO: 3 - DNA, 296 bp, Felis catus
  source 1..296: mol_type = genomic DNA
gatgtgcagctggcggaatctgggggagacctggtgaagcctggggggtccctgagactcacctgtgtggcctctggattcaccttcagtagctactacatgcactgggtccgccaggctccagggaaggggctgcagtgggtcgcacaaattagtagtagtggaggtagcacatactacgcagactccgtgaagggccgattcaccatctccagagacaacgccaagaacacgctgtatctgcagatgaacagcctgaagaccgaggacacggccacatattactgtgcgagtga

SEQ ID NO: 4 - DNA, 296 bp, Felis catus
  source 1..296: mol_type = genomic DNA
gatgtgcagctggtggaatctgggggagacctggtgaagcctggggggtccctgagactcacctgtgtagcctctggattcaccttcagtagctatgacatgaactgggtccgccaggctccagggaaggggctgcagtgggtcgcatatattagtagtagtggaggtagcatatactacgcagactccgtgaagggccgattcaccatctccagagacaacgccaagaacacgctgtatctgcagatgaacagcctgaagaccgaggacacggccacatattactgtgcgagtga

SEQ ID NO: 5 - DNA, 296 bp, Felis catus
  source 1..296: mol_type = genomic DNA
gacgagcagctggtggagtctgggggagacctggtgaagcctggggggtccctgagactcacctgtgtggcctccggattcaacgtcggtagctatggaatgagctggttccgccaggctccaggaaaggggctgcagtgggtcgcagcaattagtggtagtggaggtagcacaggttacgcagactccgtgaagggccgattcaccatctccattgacaacgccaagaacacgctgtatctgcagatggacagcctgaagactgaggacacggccacatattactgtgcgagagg

SEQ ID NO: 6 - DNA, 296 bp, Felis catus
  source 1..296: mol_type = genomic DNA
gacgtgcagctggtggagtctgggggagacctggtgaagcctggggggtccctgagactcacctgtgtagcctctggattcaccttcagtagctatgacatgagctgggtccgccaggctccagggaaggggctgcagtgggtcgcagcaattagttataatggaggtagcacaggctacgcagactccgtgaagggccgattcaccatctccagagacaacgccaagaacacgctgtatctgcagatgaacagcctcaagaccgaggacacggccacatattactgtgcgagaga

SEQ ID NO: 7 - DNA, 290 bp, Felis catus
  source 1..290: mol_type = genomic DNA
caacttacacttcgggagtccggcccaggactggtgaagccttcacaatccctctctctcacctgcgttgtctccggaggctctgttaccagcagttactactggaactggatccgccagcgccctgggagagggttggagtggctggggtactggtcaggtagcaccagctacaacccggctttccagggccgcatctccatcactgctgacacagcccagaaccagttctccctgcagctgagctccatgaccaccgaggacacggccgtgtattactgtgcaagaag

SEQ ID NO: 8 - DNA, 296 bp, Felis catus
  source 1..296: mol_type = genomic DNA
gacgtgcagctggtggagtctgggggagacctggtgaagcctggggggtccctgagactcacctgtgtggcctctggattcaccttcagtagcaatgcaatgagctgggtccgccaggctccagggaaggggctgcagtgggtctcagcaattagttatgatggaggtagcacaggctacgcagactccgtgaagggccgattcaccatctccagagacaacgccaagaacacactgtatctgcagatgaacagcctcaagaccgaggacacggccacatattactgtgcaagaga

SEQ ID NO: 9 - DNA, 317 bp, Felis catus
  source 1..317: mol_type = genomic DNA
gacgtgcagctggtggagtctgggggagacctggtgaagcctggggggtccctgagactcacctgtgtgttgcctctggattcaccttcagtagctattggattaatcagtatctattggatgagctgggtccgccaggctccaggaaaggggctgcagtgggtcgcaggaattagtggtagtggaggtagcacaggctacgcagactccgtgaagggccgattcactatctccattgacaacgccaagaacacgctgtatctgcagatggacagcctgaagactgaggacacggccacatattactgtgcgagagg

SEQ ID NO: 10 - DNA, 281 bp, Felis catus
  source 1..281: mol_type = genomic DNA
cagtcggggccgaatcaaccatcctcagtgtctggggccctgggccagagggtcactatctcctgcactggagttggtagttacgtggactggtaccaacaaatcccaggaatggcccccaaaaccatcatctatggtaatagctaccgaccctcaggggtccctgatcgattctccggctccaagtctggcagcacaggcaccctgaccatcactgggctccaggccgaagacgaggctgattattactgctcatcgtgggacagcagtggcagagctta

SEQ ID NO: 11 - DNA, 296 bp, Felis catus
  source 1..296: mol_type = genomic DNA
cagtctaggctgactcagccgccctcagtgtctggttctctgggccagagggtcaccatctcctgcgctggaagcagctctaatattggtggttatggtgtgaactggcaccaacaattcccaggaatggcccccaaaaccatcatctatggtaatagcaatcgaccctctggggtcccagatcgattctccggctccaagtctggcaacacaggcaccctgaccatcactgggctccaggctgaggacgaggctgattattactgctcatcgtgggacagcagtagcagtgctcg

SEQ ID NO: 12 - DNA, 281 bp, Felis catus
  source 1..281: mol_type = genomic DNA
cagtcggggccgaatcaaccatcctcagtgtctggggccctgggccagagggtcactatctcctgcactggaattgatagttacgtgggttggtaccaacaaatcccaggaatggcccccaaaaccatcatctatggtaatagcaaccgaccctcaggggtccctgatcgattctccggctccaagtctggcagcacaggcactctgaccatcactgggctccaggccgaggacgaggctgattattactgcgtatcgtggtacagcagtggcagagctta

SEQ ID NO: 13 - DNA, 295 bp, Felis catus
  source 1..295: mol_type = genomic DNA
cagtctgtgctgactcagccgccctcagtgtctggggccctgggccagacggtcaccatctcctgcgctggaagtgccaacaacattggtattgttggtgtgaactggtaccagcagcttccaggaaaggcccctaactcctcatctatgctaataacagacgaccctcaagtgttcctgacagattttctggctccaagtctggcaacacaggctccttgaccatcactgggctccaggctgaggacgaggctgattattactgcctgtctgctgacgttactcaaggtgctga

SEQ ID NO: 14 - DNA, 299 bp, Felis catus
  source 1..299: mol_type = genomic DNA
cagtctgtgctgactcagccgccctcagtgtctggggccctgggccagagggtcaccatctcctgcactggaagcagctccaacatcgggcgtggtaattatgtgaactggtaccaacaactctcaggaacagctcccaaactcctcatctatggtaatagcaatcgaccctcgggggtcccagatcgattttctggctccaagtctggcagcacaggctccttgaccatcactgggctgcaggctgaggacgaggctgattattactgtgcagcgtgggacagcagtctcaatgctca

SEQ ID NO: 15 - DNA, 299 bp, Felis catus
  source 1..299: mol_type = genomic DNA
cagtctgtgctgactcagccgccctcagtgtcggggtccctgggccagagggtcaccatctcctgcactggaagcagctccaacatcgggggtggtaattatgtgagctggtaccaacaagtcccaggaacggcccccaggctcctgatttatgagaataacaaacgaccctccggggtccccgatcgattctctggctccaagtctggcagctcaggctccctgaccatcactggactgcaggctgacgacgaggctgattattattgtgcatcatgggacgatagtctcagtgctca

SEQ ID NO: 16 - DNA, 281 bp, Felis catus
  source 1..281: mol_type = genomic DNA
cagtcggggccgaatcaaccatcctcagtgtctggggccctgggccagagggtcactatctcctgcactggagttggtagttacgtgggctggtaccaacaaatcccaggaatggcccccaaaaccatcatctatgataatagcaaccgaccctcaggggtccctgatcgattctccggctccaagtctggcagcacaggcaccctgaccatcactgggctccaggccgaggacgaggctgattattactgctcatcgtgggacagcagtggcagtgctta

SEQ ID NO: 17 - DNA, 288 bp, Felis catus
  source 1..288: mol_type = genomic DNA
tcctatgtgctgactcagcccccctcagtgtcggtgagcctgggacagacggccaggaccacctgtggaggaaacaacattgaaagcaaaagtgttcactggtaccagcagaagtcaggccaaacccctgtgctgattatctatagtgatagcaaccggccctcaggaatccccgaccgattctcaggcaccaactcggggaacacggccaccctgaccatcagcggggcccgggccgaggacgaggctgactattactgtcaggtgtgggacagtagtagtgatgct

SEQ ID NO: 18 - DNA, 288 bp, Felis catus
  source 1..288: mol_type = genomic DNA
tcaaacagggtgactcagcccccttccatttcagtggccctgggagagatggcaaggatcacctgtgagggaaacaacatcggaaatacatatgtttcctggtaccagcagaagccgaaccaggtgcccctgatgattatttatcaggatagcaaccggccttcagggatccctgaccgattctctggctctaactctgggaacacggccaccctgactgtcagcggggcccgggctgaggatgaggctgactattactgtctgtctgctcacagcagtagtaacgtt

SEQ ID NO: 19 - DNA, 288 bp, Felis catus
  source 1..288: mol_type = genomic DNA
tcctatgtgctgactcagcccccatcagtggcagtgaacctgggacagacggccaggatcacatgtggaggaaacaacattggaagtagttatgcttactggtaccagcagaagtcaggccaggcccctgtgctgattatctataaggatagcaaccggccctcagggatccctgaccgattctcaggcaccaactcggggaacacggccaccctgaccatcagcggggcccgggccgaggacgaggctgactattactgtcagtcatatgacagcaactatgatcct

SEQ ID NO: 20 - DNA, 278 bp, Felis catus
  source 1..278: mol_type = genomic DNA
cagtcaggactgaatcaaccatcctcagtgtctggggccctgggccagagggtcactatctcctgcactggaattggtagttacgtgggctggtaccaacaaatcccaggaatggtccccaaaaccatcatctatgataatagcaaccgaccctcaggggtccctgatcgattctccggctccaagtctggcagcacaggcactctgaccatcactgggctccaggccgaagacgaggctgattattactgctcatcgtggggcggtgacattattga

SEQ ID NO: 21 - DNA, 287 bp, Felis catus
  source 1..287: mol_type = genomic DNA
gcgatcacgatgacgcagtctccaggctccctggctgggtctccaggtcagcaggtcaccatgaactgcagggccagtcagagtgttagcagctacttagcctggtaccagcagaaaccagggcagcatcctaagctgctcatctactcagcttccacccgggcatctggagtccccgaccgattcagtggcagtgggtccgggacggatttcaccctcaccatcagcaacctccaggctgaagacgtggcgagttactactgtcagcagtattacagctctcctcc

SEQ ID NO: 22 - DNA, 302 bp, Felis catus
  source 1..302: mol_type = genomic DNA
gatattgtgatgacgcagacccctctgtccctgtccgtcacccctggagagccagcctcaatctcctgcagggccagtcagagcctcctgcacagtgatggaaatacttatctgaattggtacctgcagaagccaggccagtctccacggcgcttgatctatcttgtttccaaccgggactctggggtcccagacaggttcagtggcagcgggtcagggacagatttcaccctgagaatcagcagggtggaggctgacgacgtcggtgtttattactgcggtcaagctttacagtatcctcc

SEQ ID NO: 23 - DNA, 302 bp, Felis catus
  source 1..302: mol_type = genomic DNA
gatgtcgtgatgacgcagacccctctgtccctgcccgtcacccctggagagccggcctcaatctcctgcagggccagtcagagcctcctgcacagtaatggaaatacttatctgaattggtacctgcagaagccaggccagtctccacggctcctgatctatcaggtttccaaccgggcctctggggtcccagacaggttcagtggcagcgggtcagggacagatttcaccctgagaatcagcagggtggaggctgacgacgtcggagtttattactgccagcaagatacacatgctcctcg

SEQ ID NO: 24 - DNA, 302 bp, Felis catus
  source 1..302: mol_type = genomic DNA
gatgtcgtgatgacacagacccctctgtccctgcctgtcacccctggagagccggcctcaatctcctgcagggccagtcagagcctcctgcacagtgatggaaatacttatctgaattggtacctgcagaagccaggccagtctccacggcgactgatctataaggtttccaaccgggactctggggtcccagacaggttcagtggcagcgggtcagggacagatttcaccctgagaatcagcagggtagaggctgacgacgtcggagtttattactgcctgcaaggtacaaagtatcctac

SEQ ID NO: 25 - DNA, 302 bp, Felis catus
  source 1..302: mol_type = genomic DNA
gatgtcgtgatgacgcagacccctctgtccctgcccgtcacccctggagagccggcctcaatctcctgcagggccagtcagagcctcctgcacagtaatggaaacacctatttacattggtacctgcagaagccaggccagtctccacggcgactgatctatagggtttccaaccgggactctggggtcccagacaggttcagtggcagcgggtcagggacagatttcaccctgagaatcagcagggtggaggctgacgacgtcggagtttattactgcctgcaaggtacacacagacctcc

SEQ ID NO: 26 - DNA, 302 bp, Felis catus
  source 1..302: mol_type = genomic DNA
gatgtcgtgatgacgcagacccctctgtccctgcccgtcacccctggagagccggcctcaatctcctgcagggccagtcagagcctcctgcacagtaatggaaatacttatctgaattggtacctgcagaagccaggccagtctccacggcgactgatctataaggtttccaaccgggactctggggtcccagacaggttcagtggcagcgggtcagggacagatttcaccctgagaatcagcagggtggaggctgacgacgtcggagtttattactgccagcaaggtacacatgctcctcg

SEQ ID NO: 27 - DNA, 19 bp, Felis catus
  source 1..19: mol_type = other DNA
gtgggaacaccgatggggc

SEQ ID NO: 28 - DNA, 20 bp, Felis catus
  source 1..20: mol_type = other DNA
tcaccggctcagggaagtag

SEQ ID NO: 29 - DNA, 21 bp, Felis catus
  source 1..21: mol_type = other DNA
atgctgctgagagagtagagc

SEQ ID NO: 30 - DNA, 20 bp, Felis catus
  source 1..20: mol_type = other DNA
tggggctccaagtcactctc

SEQ ID NO: 31 - DNA, 20 bp, Felis catus
  source 1..20: mol_type = other DNA
ggttcccggaagcagacttc

SEQ ID NO: 32 - DNA, 20 bp, Felis catus
  source 1..20: mol_type = other DNA
ctgaggctcagcgggaagac

SEQ ID NO: 33 - DNA, 19 bp, Felis catus
  source 1..19: mol_type = other DNA
ggagggcttggtggtctcc

SEQ ID NO: 34 - DNA, 20 bp, Felis catus
  source 1..20: mol_type = other DNA
tcaagccgctggggtagaag

SEQ ID NO: 35 - DNA, 20 bp, Felis catus
  source 1..20: mol_type = other DNA
agggtgggaagagtgtgacc

SEQ ID NO: 36 - DNA, 21 bp, Felis catus
  source 1..21: mol_type = other DNA
ggactacgccatccactttcc

SEQ ID NO: 37 - DNA, 20 bp, Felis catus
  source 1..20: mol_type = other DNA
ggtgctgctgaggctgtagg

SEQ ID NO: 38 - DNA, 21 bp, Felis catus
  source 1..21: mol_type = other DNA
gcacacgatagaggcacttcc

SEQ ID NO: 39 - DNA, 20 bp, Felis catus
  source 1..20: mol_type = other DNA
tctcaggactggagggaagg

SEQ ID NO: 40 - DNA, 20 bp, Felis catus
  source 1..20: mol_type = other DNA
gagaaggtgacggagctggg

SEQ ID NO: 41 - DNA, 20 bp, Felis catus
  source 1..20: mol_type = other DNA
ggacagggagctctcacagg

SEQ ID NO: 42 - DNA, 20 bp, Felis catus
  source 1..20: mol_type = other DNA
gggcagtcaggaaggacagc

SEQ ID NO: 43 - DNA, 20 bp, Felis catus
  source 1..20: mol_type = other DNA
cacctgcactgactcttggg

SEQ ID NO: 44 - DNA, 20 bp, Felis catus
  source 1..20: mol_type = other DNA
tgacaccccgagaccaaggg

SEQ ID NO: 45 - DNA, 348 bp, Felis catus
  source 1..348: mol_type = genomic DNA
atggagattgtgctgggctgggttttcctggttgctcttttaaaaggtgtccagtgtgatgtgcagttggtggagtctggaggagatctggtgcagcctggggggtccctgagactcacctgtgtggcctctggattcaccttcagtagctatagcatgaactgggtccgccaggctccagggaaggggctgcagtgggtcgcatatattagatatgatggaagtagcacagcctacgcagactccgtgaagggccgattcaccatctccagagacaacgccaagaacacgctgtatctgcagatgaacagcctcaagaccgaggacacggccacatattactgtgcg

SEQ ID NO: 46 - DNA, 353 bp, Felis catus
  source 1..353: mol_type = genomic DNA
atggcccggttccctcttctcctgacccttctcatccactgcacaggttcctgggcccagtctgtgctgactcagccgccctcagtgtctggggccctgggccagacggtcaccatctcctgcgctggaagtgccaacaacattggtattgttggtgtgaactggtaccagcagcttccaggaaaggcccctaaactcctcatctatgctaataacagacgaccctcaagtgttcctgacagattttctggctccaagtctggcaacacaggctccttgaccatcactgggctccaggccgaggacgaggctgattattactgcatgtctgctgacattactcaaggtgctga

SEQ ID NO: 47 - DNA, 338 bp, Felis catus
  source 1..338: mol_type = genomic DNA
atggcctggtcccctctcctcctcactctcctcgctcactgcacagggatttgggctcagtcaggactgaatcaaccatcctcagtgtctggggccctgggccagagggtcactatctcctgcactggagctggtagttacgtgggctggtaccaacaaatcccaggaatggcccccaaaaccatcatctatgataatagcaaccgaccctcaggggtccctgatcgattctccggctccaagtctggcagcacaggcaccctgaccatcactgggctccaggccgaggacgaggctgattattactgctcatcgtgggacagcagtggcacagctta

SEQ ID NO: 48 - DNA, 31 bp, Felis catus
  source 1..31: mol_type = genomic DNA
gtgactactgtatcgggagtagctgtgctac